(19) United States
(12) Patent Application Publication
(10) Pub. No.: US 2024/0158560 A1
(43) Pub. Date: May 16, 2024

(54) POLYMER CAPABLE OF FORMING ELASTIC MATERIAL BY THERMAL CROSSLINKING, PREPARATION METHOD THEREFOR, AND APPLICATION THEREOF

(71) Applicant: INNOPOLY BIOTECH (HANGZHOU) INC., Hangzhou (CN)

(72) Inventors: Yonghua ZHOU, Hangzhou (CN); Yunbing WANG, Chengdu (CN)

(21) Appl. No.: 18/479,537

(22) Filed: Oct. 2, 2023

Related U.S. Application Data
(63) Continuation of application No. PCT/CN2021/085444, filed on Apr. 2, 2021.

(51) Int. Cl. C08F 297/04; A61L 27/16; A61L 27/50

(52) U.S. Cl. CPC C08F 297/04; A61L 27/16; A61L 27/50; A61L 2430/20; C08F 2810/20

(57) ABSTRACT

A polymer for heart valve prosthesis that is capable of forming elastic material by thermal crosslinking is disclosed. The elastic material includes polymers A used as hard segments and a polymer B used as a soft segment, and the chemical formula is: (Aₘ)ᵢ(Bₙ)ⱼ(A_{f})ₖ or (Aₘ−Bₙ)ₚX(Bₙ−A_{f})_{q}; the polymers A are polymers formed by polymerization of at least one of vinyl aromatic hydrocarbon and a thermally crosslinking monomer, or polymers formed by copolymerization of at least one of vinyl aromatic hydrocarbon and the thermally crosslinking monomer and conjugated diene; the polymer B is a conjugated diene polymer, or a polymer formed by copolymerization of at least one of vinyl aromatic hydrocarbon and the thermally crosslinking monomer and conjugated diene; at least one of the polymer A and the polymer B contains the thermally crosslinking monomer.

### CROSS-REFERENCE TO RELATED APPLICATIONS

The present application is a Continuation Application of PCT Application No. PCT/CN2021/085444, filed on Apr. 2, 2021, the entire contents of which are hereby incorporated by reference.

### TECHNICAL FIELD

The present disclosure relates to the field of biomedical materials, in particular to polymers capable of forming elastic material by thermal crosslinking, preparation method therefor, and application thereof.

### DESCRIPTION OF THE PRIOR ART

Biomedical materials are materials used to diagnose, treat, repair or replace diseased tissues or organs of organisms, or enhance the functions thereof. Implantable materials for human tissue replacement and repair usually include two elastic materials: polyurethane and silicone rubber. Although polyurethane and silicone rubber are widely used in a variety of implantable medical devices, such as breast prostheses, cardiac pacemakers, artificial blood vessels, intraocular lenses, joint prostheses, heart valves, etc., such two elastic materials have problems of degradation and calcification after being implanted into human body for a long time [Biomaterials 2008; 29: 448-460] [Int. J. Biomed. Eng. 2014; 37: 57-60].

In the mid-1990s, a new elastic material, SIBS, a thermoplastic elastomer based on polystyrene-polyisobutylene-polystyrene triblock polymers, was found to have biological inertness, excellent biological stability and compatibility [Biomaterials 2008; 29: 448-460]. This material has been used for several Class III medical devices including drug-loaded coatings for cardiovascular stents and glaucoma drainage tubes. Nearly 20 years of clinical practice has confirmed that this material has no degradation, calcification and foreign body reactions that are common for polyurethanes and silica gel materials. The biological inertness of SIBS attributes to its molecular structure and composition: the SIBS polymer synthesized by active cationic polymerization has a narrow molecular weight distribution and does not contain residual monomers, oligomers and small molecule additives and the like that are likely to cause foreign body reactions; the polymer is composed of two blocks of polystyrene and polyisobutylene so that only two chemical bonds of carbon-hydrogen and carbon-carbon are presented in the molecular structure, which is not easy to be degraded in the human body to lose its function or release small molecules that cause foreign body reactions.

As SIBS material is a thermoplastic elastomer, it will creep and thus deform under long-term stress, which limits its biomedical application. Yonghua Zhou, one of the inventors of the present invention, invented a thermally crosslinkable SIBS material (i.e., XSIBS material) as described in U.S. Pat. No. 8,765,895. This material can be cross-linked when heated without additive catalysts, crosslinking agents, etc, in which cross-linking process no small molecules such as water, alcohol, acid, etc., are released, so it has the same biological stability and biocompatibility as SIBS material. XSIBS material has currently been used for the development of heart valve prostheses [Annals Biomed Engi. 2019; 47:113-125]. Further, Yonghua Zhou, one of the inventors of the present invention, invented a thermally crosslinkable polyisobutylene derived from XSIBS material as described in U.S. Pat. No. 8,585,940, which does not contain polystyrene but is thermally crosslinkable, and is used to develop a new generation of intraocular lens.

Hydrogenated styrene-based block polymer (HSBC) is a block polymer material similar to SIBS, which has the characteristics of thermoplastic elastomer, that is, it can be processed as easily as thermoplastic and also has the same elasticity as thermosetting rubber. What thermoplastic elastomers such as HSBC and SIBS have in common is that they are both multi-block polymers, including hard segments of polymers such as polystyrene, and soft segments of polymers such as polyisobutylene or hydrogenated polybutadiene or hydrogenated polyisoprene. The hard segments are the dispersed phase, located at the two ends of the polymers, and the soft segments are the continuous phase, located in the middle of the polymers, so that the dispersed hard segments with the continuous soft segments form physical crosslinks providing the material with rubber elasticity and thermoplastic processability which allows the material to be melt- or solution-processed.

The fundamental difference between HSBC and SIBS is that: SIBS is synthesized by active cationic polymerization of styrene and isobutylene, and the middle rubber segment in the molecular structure is composed of saturated polyisobutylene; while HSBC is synthesized by active anionic polymerization of styrene and conjugated diene, followed by selective hydrogenation to saturate the double bonds on the polyconjugated diene. The first step of the synthesis of HSBC is to obtain a polystyrene-polyconjugated diene-polystyrene triblock polymer by anionic polymerization, which is a thermoplastic elastomer, while each monomer unit of polyconjugated diene in the rubber segment contains a double bond formed during polymerization, which is unstable at high temperature or in an oxidative environment. The second step of the synthesis of HSBC is to use a catalyst for selective hydrogenation to convert the double bond of the polyconjugated diene into a saturated carbon-carbon bond, thereby solving the instability problem due to the unsaturated bond. Conjugated diene generally includes butadiene and isoprene. Commercial HSBC polymers mainly include SEBS and SEPS, wherein SEBS uses butadiene monomer, and SEPS uses isoprene monomer.

Active anionic polymerization is a real active polymerization, while active cationic polymerization is a controllable active polymerization. HSBC material synthesized by anionic polymerization has a very narrow molecular weight distribution (molecular weight polydispersity index is generally lower than 1.1). However, SIBS material obtained by cationic polymerization has a wide molecular weight distribution in the final product as the styrene polymerization is difficult to control and also accompanied by coupling reactions (molecular weight polydispersity index is generally about 1.3, and a small amount of coupling products are usually generated in the later stage of polymerization). Therefore, HSBC is a purer and single block polymer with better mechanical properties (such as higher tensile strength).

In addition, HSBC has greater flexibility in molecular structure design than SIBS. The monomer for HSBC rubber phase can be selected from isoprene, butadiene and a mixture of the two, while the rubber monomer of SIBS can be only isobutylene. The rubber phase of HSBC can be obtained through random copolymerization by introducing styrene and conjugated diene monomers, with the resulted rubber phase containing styrene monomer units. The rubber phase of HSBC can greatly improve the mechanical properties (such as tensile modulus, abrasion resistance and tear resistance) of the elastomer by introducing styrene monomer units through random copolymerization, approaching the properties of polyurethane elastomer, thereby expanding its application range [U.S. Pat. No. 7,169,848]. For SIBS, because the random copolymerization of styrene and isobutylene is difficult to achieve, it is difficult to improve the mechanical properties by introducing styrene into the rubber phase. Therefore, it is difficult for SIBS to approach the unique properties of polyurethane, and the application range is greatly limited.

HSBC material is an ideal medical material because it has the following advantages: no plasticizers and allergens, very low amount of extracts and filtrates, no hydrolysis or degradation, no stimulation to the human body, easy processing and shaping, and suitable for various disinfection methods (ethylene oxide, gamma rays, electron beams, ultraviolet rays, high temperature), etc. [https://kraton.com/products/pdf/Medicalc/020Brochure.pdf]. HSBC material can pass relevant important medical standard tests, such as ISO10993 biocompatibility test and USP Class VI certification. Biomedical applications of HSBC material are currently limited to lower-risk medical devices (Classes I and II) or consumables. In the medical field, HSBC is generally mixed with other components (such as polyolefin, polyurethane, engineering plastics, mineral oil, etc.), and then processed into medical products (such as infusion tubes, infusion bags, syringes, seals, medical connectors, stoppers and caps for medicine bottles, medical packaging, wound bandages, skin patches, surgical drapes, medical gowns, etc.). Although these devices can be implanted into human body, they are limited to within 30 days, and there is no long-term implantation application in the human body yet. This is just the opposite of SIBS material. SIBS has been applied for three types of medical devices that can be implanted in the human body for a long time, such as cardiovascular stents and glaucoma drainage tubes, but is rarely applied for low-risk, short-term implantable medical devices or consumables due to high price.

Both HSBC and SIBS are non-hydrolyzable hydrocarbons and do not contain biotoxic small molecules of extracts and filtrates, and thus have good biocompatibility. In the aspect of material composition, the only substantial difference between HSBC and SIBS lies in the monomer composition of the rubber phase in the molecular structure. The rubber phase of SIBS includes polyisobutylene, while the rubber phase of HSBC mainly includes copolymers of ethylene and 1-butene or copolymers of ethylene and propylene (or hydrogenated polybutadiene or hydrogenated polyisoprene). The biological stability of SIBS is attributed to the molecular structure of polyisobutylene, which has no hydrogen atoms that are easy to be abstracted for degradation reaction and thus is completely biologically inert [U.S. Pat. No. 6,102,939]. However, in fact, SIBS material is prone to degradation under the irradiation of ultraviolet rays, gamma rays, electron beams, etc., and thus SIBS is generally suitable for disinfection with ethylene oxide, while HSBC is more stable under these rays and can be disinfected with these rays, which means that HSBC may have better stability in the human body, at least can be implanted in human body for a long time like SIBS. In fact, HSBC should have a wider range of biomedical applications than SIBS due to its superior mechanical properties.

In summary, HSBC, as an implantable long-term material, can not only overcome the deficiencies of polyurethane and silica gel (easy to degrade and calcify, etc.), but also overcome the deficiencies of SIBS material in the mechanical properties. Therefore, HSBC can replace polyurethane, silica gel and SIBS and be applied for many implantable long-term medical devices, including intraocular lenses, heart valves, pacemaker wire insulation materials, artificial blood vessels, glaucoma drainage tubes, cosmetic materials, etc. However, HSBC, like SIBS, is a thermoplastic elastomer, which will creep or permanently deform under long-term stress and lose its proper function. If HSBC is chemically cross-linked without introducing biotoxic additives such as catalysts and releasing biotoxic small molecules, then it can be applied for medical devices under long-term stress with lower deformation and failure.

Cataract is a primary blinding disease of human beings. Various kinds of reasons, such as aging, genetics, local dystrophies, immune and metabolic abnormalities, trauma, poisoning, radiation, etc., can cause lens metabolic disorders, resulting in lens protein denaturation and opacification, thus cataract occurs. Although drugs can relieve or ameliorate cataracts at the early and middle stages, a more effective treatment method is to remove the cloudy lens nucleus through surgery, and implant an intraocular lens to restore the patient's vision. The earliest intraocular lens material used by human beings is glass, which has the disadvantages of being relatively heavy and fragile during the surgery. Later, organic glass (polymethyl methacrylate) was used, which has the disadvantages of being very hard and difficult to fold, requiring a large incision (about 6 mm in length) for implantation that will need to be sutured, which will cause great damage to the patient's eye and the postoperative recovery period will be longer. With the development and application of laser emulsification, the incision required for lens extraction has become smaller and smaller (about 2 mm). Also, softer and easier-to-fold intraocular lenses have been successfully developed, which can be implanted into the capsule through a small surgical incision. Such minimally invasive implantation surgery does not require suture, and patients do not need to be hospitalized, but can usually leave the hospital within a few hours after the surgery.

Materials currently used for foldable intraocular lenses can be divided into three categories: silicone rubber, hydrophilic acrylates, and hydrophobic acrylates [Turk J Ophthalmol 2017; 47:221-225] [Medicine 2017; 96:44] [e-Polymers 2009; 9(1): 1466]. The main disadvantages of silicone rubber are: insufficient biocompatibility, which results in calcification and inflammation after long-term implantation; low refractive index, which requires a relatively thick intraocular lens; poor mechanical strength such as weak tear resistance, poor tensile property and high tensile modulus, which is not conducive to the implantation of the folded lens; quick absorption of silicone oil, which will affect the optical effect, while silicone oil is often used as filling material after vitrectomy; being prone to YAG laser damage, while YAG laser is often used for the treatment of after-cataract; easy to damage the capsule, as when the folded lens is unfolded, it usually restores to its original shape quickly and elastically. Due to these factors, commercial intraocular lenses are usually made of acrylate materials including hydrophilic and hydrophobic acrylates. However, the hydrophilic acrylates involve the risk of after-cataract and calcification and have low refractive index, while hydrophobic acrylates also have deficiencies, such as poor mechanical properties (e.g. tear resistance and tensile properties) and thus easy to break during folding and implantation, and prone to flare and leucoma after implantation.

In order to overcome the shortcomings of the existing intraocular lenses, a technology of synthesis of thermally crosslinkable polyisobutylene material by cationic polymerization was developed (U.S. Pat. No. 8,765,895), which has been used to make a new type of intraocular lens (U.S. Pat. No. 8,585,940). This intraocular lens material has advantages of: excellent biocompatibility and biological stability; one-time molding or injection molding without any subsequent processes of solvent extraction and purification; no flare and leucoma; being soft, with low tensile modulus and high breaking elongation, easy to fold and implant; higher refractive index and Abbe index than silica gel. Currently, this technology is still in the development stage, and its advantages for making a new type of intraocular lens have yet to be clinically verified. Further, this technology has several shortcomings: the glass transition temperature of polyisobutylene is far lower than room temperature, so the cross-linked material based on polyisobutylene, being folded and implanted, will unfold very quickly and damage the capsule; the polymerization process is cationic polymerization at ultra-low temperature, the polymer washing process is particularly complicated, the material is a viscous liquid and is not easy to process, and the chlorine component contained in the material will corrode the mold during hot pressing, which may make the cost of the intraocular lens high; the tensile strength and breaking elongation of the material are still low, although they are higher than those of acrylates, which limits the development in high-end intraocular lens technologies (such as intraocular lens requiring ultra-small incision, intraocular lens with adjustable focus, etc.).

Therefore, new intraocular lens materials and products are still needed to overcome the various deficiencies of the existing intraocular lens technologies.

Heart valves refer to the valves between the atrium and the ventricle or between the ventricle and the artery, including the mitral valves between the left ventricle and the left atrium, the tricuspid valve between the right ventricle and the right atrium, the aortic valve at the left ventricular outflow tract and the pulmonary valve at the right ventricular outflow tract. When the structure or function of the heart valve changes, the blood cannot be discharged smoothly or the discharged blood flows back, which increases cardiac load, thereby causing a series of diseases, i.e., so called valvular heart disease. There are many causes of valvular heart disease, mainly include rheumatic and degenerative. Rheumatic valvular heart disease often occurs repeatedly due to rheumatic fever, resulting in deformation of the heart valve, causing valvular stenosis or insufficiency; rheumatic valvular heart disease usually occurs at the age of 20 to 40, mostly involving the mitral valve, then the aortic valve, or the mitral, aortic and tricuspid valves at the same time, and rarely involve the pulmonary valve. Degenerative valvular heart disease is mostly caused by valvular calcification in the elderly population (generally over 60 years old), manifested as valve thickening, hardening and deformation, and calcification, etc., leading to valvular stenosis or insufficiency, mostly involving the aortic valve, or the mitral valve with degenerative insufficiency or stenosis.

For patients with severely calcified or damaged valves, prosthetic valve replacement is the most effective treatment method. Early prosthetic valve replacement requires surgical thoracotomy. Prosthetic valves mainly include two types: mechanical valves and biological valves. Mechanical valves are generally made of artificial materials such as titanium, graphite matrix, and pyrolytic carbon. Although mechanical valves have good durability, patients need to take anticoagulant drugs for life. Biological valves are usually artificially processed from bovine or horse pericardium or porcine valves, which have more similar characteristics to human physiological valves; although biological valves have good anticoagulation properties and patients do not need to take anticoagulant drugs for a long time, their durability is only 5-10 years due to aging and gradual wear. In recent years, transcatheter heart valve replacement has become the mainstream of heart valve replacement technologies because of less surgical injury [Ann Cardiothorac Surg (2017) 6(5):493-7]. Since mechanical valves cannot be implanted through the catheter, such interventional methods are only suitable for soft, foldable biological valves. However, in addition to the poor durability, the raw materials of biological valves (animal pericardia) are difficult to keep the consistency due to individual differences in animal-derived materials, and the cost is high, difficult to achieve mass production; in addition, biological valves may even carry animal-derived diseases and endanger patients, and even lead to death.

Therefore, clinically, there is an urgent need for prosthetic valve materials with better performances, which not only have the durability of mechanical valves and the biocompatibility of biological valves, but also can be implanted through minimally invasive intervention like biological valves, and can also overcome the deficiencies of biological valves (high cost, difficult to control the quality of raw materials among others).

The polymer materials for making heart valve prostheses has several obvious advantages: 1) polymer materials can be mass-produced, and their performances and quality can be stably controlled, thereby greatly reducing costs; 2) polymer materials have various mechanical properties, which can be designed in molecular structure and chemical composition to achieve the performances required for heart valve products (including soft and foldable properties to meet the requirements for minimally invasive surgery such as transcatheter intervention); 3) polymer materials can be processed through different methods to obtain heart valves of different sizes and shapes, while biological valves can only be cut and sewed and cannot be reprocessed to change the thickness and shape in most situations, which makes the valve design can be optimized through biomechanical analysis and finite element analysis; 4) polymer materials generally do not carry diseases of animal origin.

Despite these advantages, polymer materials still have challenges in terms of biocompatibility, durability, and fatigue resistance, in order to be applied in heart valve prosthesis products. Polymeric materials have been developed for decades to make prosthetic heart valves, but have no successful clinical applications yet [Biomaterials 36 (2015) 6-25]. Polymer materials used to make heart valve prostheses include silica gel, expanded polytetrafluoroethylene, polyurethane, SIBS (styrene-isobutylene-styrene triblock polymer), ethylene-propylene rubber, and polyvinyl alcohol hydrogel. The mechanical properties of silica gel and expanded polytetrafluoroethylene cannot meet the requirements of heart valves, while polyurethane materials cannot meet the durability requirements due to poor hydrolytic stability. SIBS (styrene-isobutylene-styrene triblock polymer) is an elastic material with excellent biocompatibility and biological stability, but it is prone to creep and deform under long-term force due to its thermoplastic characteristics. Heart valves made of SIBS materials reinforced by polyester, in sheep animal model experiments, show calcification and coagulation due to exposure of embedded polyester as the polymer creeps [Wang, Q. et al, 2010, “In-Vivo Assessment of a Novel Polymer (SIBS) Trileaflet Heart Valve,” J. Heart Valve Dis., 19(4), pp. 499-505]. Thermally crosslinked SIBS material (XSIBS) has creep resistance, and the heart valve made of this material has improved hemodynamic and anticoagulation properties, but it is still in the stage of laboratory development and has not yet entered clinical application. Neither ethylene-propylene rubber nor polyvinyl alcohol hydrogel has been reported on clinical trials, let alone product registration and commercialization.

Biological valve materials are generally derived from animal pericardial tissue, and have a natural three-dimensional scaffold structure composed of collagen, elastin, fibronectin and laminin. These pericardial tissue materials are cross-linked and anti-calcification treated, and then sewn into heart valve products. Compared with polymer elastic materials, biological valve materials are soft but have very low elongation (about 10-20%). In order to allow polymer materials to have the mechanical properties of biological valve materials, fiber reinforcement is a commonly used method. Fiber reinforcement has been applied on silica gel, polyurethane, and SIBS materials, and usually uses polyester, but due to the deficiencies of the polymer materials, no heart valve products have been successfully developed for clinical applications. One of the deficiencies of polyester is that it is not resistant to high temperatures and easily deformed and brittle at high temperatures above 200° C., losing its function of reinforcing polymer materials, and thus it is not suitable for processes that require high temperature to make composite materials.

### SUMMARY OF THE DISCLOSURE

In view of this, the present invention provides a polymer capable of forming elastic material by thermal crosslinking and synthesized by anionic polymerization and a preparation method thereof. The polymer capable of forming elastic material by thermal crosslinking can be chemically crosslinked by heating to obtain a medical device or a part of the medical device suitable for long-term implantation in the human body.

A polymer capable of forming elastic material by thermal crosslinking and synthesized by anionic polymerization is provided, the elastic material is a saturated block copolymer, including polymer A as hard segment and polymer B as soft segment, with a chemical formula of:

(Aₘ)ᵢ(Bₙ)ⱼ(A_{f})ₖ or (Aₘ−Bₙ)ₚX(Bₙ−A_{f})_{q};
compositions of polymers A at two ends of the polymer B are independent of each other;
polymer A is a polymer formed by polymerization of at least one of vinyl aromatic hydrocarbon and thermally crosslinking monomer, or a polymer formed by copolymerization of at least one of vinyl aromatic hydrocarbon and thermally crosslinking monomer with conjugated diene;
polymer B is a conjugated diene polymer, or a polymer formed by copolymerization of at least one of vinyl aromatic hydrocarbon and thermally crosslinking monomer with conjugated diene;
at least one of the polymer A and the polymer B contains the thermally crosslinking monomer;
X represents a residual group of a coupling agent after coupling reaction;
subscripts m and f each represents a number of comonomer units of the polymer A, subscript n represents a number of comonomer units of the polymer B, and m, n, f are all integers greater than or equal to 1 and independent of each other;
subscripts i and k each represents a number of blocks of the polymer A, subscript j represents a number of blocks of the polymer B, both i and k are integers greater than or equal to 0, j is an integer greater than or equal to 1, and i, j, k are independent of each other;
subscripts p and q each represents a number of blocks formed by polymerization of the polymer A and the polymer B, and both p and q are integers greater than or equal to 0 and are independent of each other;
the thermally crosslinking monomer has a chemical structural formula of:

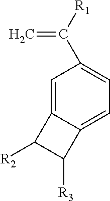

wherein, R₁, R₂ and R₃ are each hydrogen or C₁-C₁₀ alkyl group, and are independent of each other.

The thermally crosslinkable elastic material is generally a block copolymer. Both polymer A and polymer B are random copolymers formed by anionic polymerization, and polymer A and polymer B are respectively blocks of the block copolymer. The subscripts i and k represent the numbers of blocks of polymer A, and the subscript j represents the number of blocks of polymer B. The subscripts satisfy i+k=j, or i+k=j+1.

For example: when i, j, k are 1 respectively, the chemical formula of the elastic material is ABA.

When i and k are 1 and j is 2, the chemical formula of the elastic material is ABAB.

When i is 1, k is 2, and j is 3, the chemical formula of the elastic material is ABABAB.

The numbers of comonomer units of the blocks of the block copolymer can be the same or different. m, n, and f only represent the numbers of comonomer units of polymer A and polymer B. In terms of a more complicated block copolymer, such as ABAB, the numbers of comonomer units of the two polymers A as blocks can be different, similarly, the numbers of comonomer units of the two polymers B as blocks can also be different.

The polymer capable of forming elastic material by thermal crosslinking is a saturated block copolymer, therefore, polymer A and polymer B are also saturated polymers. If polymer A and polymer B remain unsaturated double bonds after polymerization, they can be converted into saturated structures by using existing techniques, such as catalytic hydrogenation. That is, polymer A is a polymer formed by polymerization of at least one of vinyl aromatic hydrocarbon and thermally crosslinking monomer, or a saturated polymer formed by hydrogenation after copolymerization of at least one of vinyl aromatic hydrocarbon and thermally crosslinking monomer with conjugated diene.

Polymer B is a saturated polymer formed by hydrogenation after polymerization of conjugated diene; or a saturated polymer formed by hydrogenation after copolymerization of at least one of vinyl aromatic hydrocarbon and thermally crosslinking monomer with conjugated diene.

Unless otherwise specified, the unsaturated double bond structure after the polymerization of the conjugated diene is converted into a saturated structure by catalytic hydrogenation, as a component of the polymer capable of forming elastic material by thermal crosslinking.

The following further provides several options. They are not used as additional limitations on the above-mentioned subject matter, but are only further additions or preferences. Without technical or logical contradiction, the options can be combined with the above-mentioned subject matter independently or in combination.

Optionally, at least one of the polymer A and the polymer B contains the vinyl aromatic hydrocarbon.

Optionally, the polymer A is the polymer formed by polymerization of at least one of vinyl aromatic hydrocarbon and thermally crosslinking monomer, or the polymer formed by copolymerization of at least one of vinyl aromatic hydrocarbon and thermally crosslinking monomer with conjugated diene;
the polymer B is the conjugated diene polymer, or the polymer formed by copolymerization of at least one of vinyl aromatic hydrocarbon and thermally crosslinking monomer with conjugated diene; and
when the polymer A is the polymer formed by polymerization of vinyl aromatic hydrocarbon and thermally crosslinking monomer, the polymer B is the polymer formed by copolymerization of at least one of vinyl aromatic hydrocarbon and thermally crosslinking monomer with conjugated diene.

Optionally, the polymer A is the polymer formed by polymerization of at least one of vinyl aromatic hydrocarbon and thermally crosslinking monomer, and at least one segment of the polymer A of the elastic material contains thermally crosslinking monomer.

At least one segment of the polymer A of the elastic material containing thermally crosslinking monomer means, for example, when the structural formula of the elastic material is ABA, at least one block of polymer A contains thermally crosslinking monomer.

Optionally, the polymer A is the polymer formed by polymerization of at least one of vinyl aromatic hydrocarbon and thermally crosslinking monomer, or the polymer formed by copolymerization of at least one of vinyl aromatic hydrocarbon and thermally crosslinking monomer with conjugated diene; and
the polymer B is the polymer formed by copolymerization of at least one of vinyl aromatic hydrocarbon and thermally crosslinking monomer with conjugated diene.

Optionally, the polymer A is the polymer formed by polymerization of at least one of vinyl aromatic hydrocarbon and thermally crosslinking monomer, and at least one segment of the polymer A of the elastic material contains thermally crosslinking monomer; and
the polymer B is the polymer formed by copolymerization of at least one of vinyl aromatic hydrocarbon and thermally crosslinking monomer with conjugated diene.

Optionally, the polymer A is a polymer of the vinyl aromatic hydrocarbon, or a copolymer of the vinyl aromatic hydrocarbon and the thermally crosslinking monomer, and at least one segment of the polymer A of the elastic material contains thermally crosslinking monomer; and
the polymer B is the polymer formed by copolymerization of at least one of vinyl aromatic hydrocarbon and thermally crosslinking monomer with conjugated diene.

Optionally, the polymer A is a copolymer of the vinyl aromatic hydrocarbon and the thermally crosslinking monomer; and
the polymer B is the polymer formed by copolymerization of at least one of vinyl aromatic hydrocarbon and thermally crosslinking monomer with conjugated diene.

Optionally, m, n, f are all integers greater than or equal to 10.

Optionally, both i and k are integers greater than or equal to 0 and less than or equal to 30, and i and k are not 0 at the same time. j is an integer greater than or equal to 1 and less than or equal to 30.

Optionally, both p and q are integers greater than or equal to 0 and less than or equal to 30, and p and q are not 0 at the same time.

Optionally, the chemical structural formula of the thermally crosslinking monomer is:

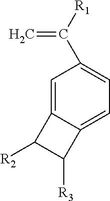

wherein, R₁, R₂ and R₃ are each hydrogen or C₁-C₅ alkyl group, and are independent of each other.

Optionally, the chemical structural formula of the thermally crosslinking monomer is:

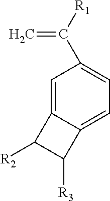

wherein, R₁, R₂ and R₃ are each hydrogen or C₁-C₃ alkyl group, and are independent of each other.

Optionally, the chemical structural formula of the thermally crosslinking monomer is:

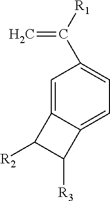

wherein, R₁, R₂ and R₃ are each hydrogen or methyl or ethyl, and are independent of each other.

Optionally, the chemical structural formula of the thermally crosslinking monomer is:

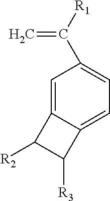

wherein, R₁, R₂ and R₃ are each hydrogen.

When R₁, R₂ and R₃ are each hydrogen, the thermally crosslinking monomer is 4-vinyl benzocyclobutene.

Optionally, the vinyl aromatic hydrocarbon contains at least one vinyl group and at least one aromatic group, and a conjugation effect is presented between the at least one vinyl group and the at least one aromatic group.

Unless otherwise specified, vinyl group in the present disclosure refers to a group containing a carbon-carbon double bond, and the carbon atoms involved in the carbon-carbon double bond can contain substituent(s) which can be methyl, ethyl, propyl, butyl and other alkyl groups.

Optionally, the vinyl aromatic hydrocarbon contains one aromatic group and at least one vinyl group, and a conjugation effect is presented between the aromatic group and the at least one vinyl group.

Optionally, the vinyl aromatic hydrocarbon contains one vinyl group and at least one aromatic group, and a conjugation effect is presented between the vinyl group and the at least one aromatic group.

Optionally, the vinyl aromatic hydrocarbon contains one aromatic group and one vinyl group, and a conjugation effect is presented between the vinyl group and the aromatic group.

Optionally, the vinyl aromatic hydrocarbon contains one aromatic group and two vinyl groups, and a conjugation effect is presented between the aromatic group and at least one vinyl group.

Optionally, the vinyl aromatic hydrocarbon is at least one of styrene, α-methylstyrene, 4-methylstyrene, vinylnaphthalene, 1,1-diphenylethene and divinylbenzene.

Optionally, the vinyl aromatic hydrocarbon is styrene.

The conjugated diene in this disclosure contains at least two carbon-carbon double bonds with a conjugation effect therebetween, and the carbon atoms involved in the carbon-carbon double bond can contain substituent(s) which may be methyl, ethyl, propyl, butyl and other alkyl groups.

Optionally, the conjugated diene is at least one of isoprene, 1,3-butadiene, 1,3-pentadiene, 4-methylpentadiene, and 2-methylpentadiene.

Optionally, the conjugated diene is at least one of isoprene and 1,3-butadiene.

Optionally, the conjugated diene is 1,3-butadiene.

Optionally, the conjugated diene is isoprene.

Optionally, the vinyl aromatic hydrocarbon is styrene, the conjugated diene is at least one of isoprene and 1,3-butadiene, and the thermally crosslinking monomer is 4-vinylbenzocyclobutene.

Optionally, the vinyl aromatic hydrocarbon is styrene, the conjugated diene is isoprene or 1,3-butadiene, and the thermally crosslinking monomer is 4-vinylbenzocyclobutene.

Optionally, the vinyl aromatic hydrocarbon is styrene, the conjugated diene is isoprene, and the thermally crosslinking monomer is 4-vinylbenzocyclobutene.

Optionally, the vinyl aromatic hydrocarbon is styrene, the conjugated diene is 1,3-butadiene, and the thermally crosslinking monomer is 4-vinylbenzocyclobutene.

Optionally, the vinyl aromatic hydrocarbon is α-methylstyrene, the conjugated diene is at least one of isoprene and 1,3-butadiene, and the thermally crosslinking monomer is 4-vinyl benzocyclobutene.

Optionally, the vinyl aromatic hydrocarbon is α-methylstyrene, the conjugated diene is isoprene or 1,3-butadiene, and the thermally crosslinking monomer is 4-vinylbenzocyclobutene.

Optionally, the vinyl aromatic hydrocarbon is α-methylstyrene, the conjugated diene is isoprene, and the thermally crosslinking monomer is 4-vinylbenzocyclobutene.

Optionally, the vinyl aromatic hydrocarbon is α-methylstyrene, the conjugated diene is 1,3-butadiene, and the thermally crosslinking monomer is 4-vinylbenzocyclobutene.

Optionally, the content of the thermally crosslinking monomer in the polymer A is 0-99.99%.

Optionally, the content of the thermally crosslinking monomer in the polymer A is 0.01-99.99%.

Optionally, the content of the thermally crosslinking monomer in the polymer A is 0.1-5%.

Optionally, the content of the thermally crosslinking monomer in the polymer A is 1-2%.

Optionally, the content of the thermally crosslinking monomer in the polymer B is 0.01-80%.

Optionally, the content of the thermally crosslinking monomer in the polymer B is 0.1-5%.

Optionally, the content of the thermally crosslinking monomer in the polymer B is 1-2%.

Optionally, the content of the conjugated diene in the polymer A is 0-50%.

Optionally, the content of the conjugated diene in the polymer B is 0.01-100%.

Optionally, the content of the vinyl aromatic hydrocarbon in the polymer A is 60-100%.

Optionally, the content of the vinyl aromatic hydrocarbon in the polymer A is 90-100%.

Optionally, the content of the vinyl aromatic hydrocarbon in the polymer A is 95-100%.

Optionally, the content of the vinyl aromatic hydrocarbon in the polymer B is 0-70%.

Optionally, the content of the thermally crosslinking monomer in the thermally crosslinkable elastic material is 0.05-10%, the content of the conjugated diene is 30-90%, and the remaining is vinyl aromatic hydrocarbon.

Optionally, the content of the thermally crosslinking monomer in the thermally crosslinkable elastic material is 0.1-5%, the content of the conjugated diene is 30-90%, and the remaining is vinyl aromatic hydrocarbon.

Optionally, the content of the thermally crosslinking monomer in the thermally crosslinkable elastic material is 0.1-2%, the content of the conjugated diene is 40-90%, and the remaining is vinyl aromatic hydrocarbon.

In the present disclosure, the content refers to the content of the respective monomer unit in the polymer after the polymerization is completed. For example, the content of the vinyl aromatic hydrocarbon is the weight percentage of the vinyl aromatic hydrocarbon monomer unit in the polymer.

Optionally, the glass transition temperature of polymer A is higher than 80° C., and the glass transition temperature of polymer B is lower than 35° C.

Optionally, the glass transition temperature of polymer A is higher than room temperature, and the glass transition temperature of polymer B is lower than room temperature.

In the present disclosure, the glass transition temperature refers to the glass transition temperature measured by DSC after the polymer capable of forming elastic material by thermal crosslinking is cross-linked.

Optionally, the molecular weight of the polymer capable of forming elastic material by thermal crosslinking is 5000-1000000.

Optionally, the molecular weight of the polymer capable of forming elastic material by thermal crosslinking is 10000-500000.

Optionally, the molecular weight of the polymer capable of forming elastic material by thermal crosslinking is 10000-150000.

Optionally, the molecular weight of the polymer capable of forming elastic material by thermal crosslinking is 80000-150000.

In the present disclosure, the molecular weight refers to the relative number average molecular weight measured by GPC and calibrated with polystyrene standard.

Optionally, the molecular weight distribution of the polymer capable of forming elastic material by thermal crosslinking is 1.0-1.3.

Optionally, the molecular weight distribution of the polymer capable of forming elastic material by thermal crosslinking is 1.0-1.1.

The polymer capable of forming elastic material by thermal crosslinking is transformed into a thermosetting elastic material at high temperature, and the thermosetting elastic material is used to prepare a medical device implantable in the human body, or a part of a medical device implantable in the human body.

The polymer capable of forming elastic material by thermal crosslinking provided by the invention is synthesized by active anionic polymerization, and does not contain small molecular additives or residual components. The elastic material has no unstable double bonds in the molecular structure after selective catalytic hydrogenation, and thus has excellent high-temperature-oxidation resistance and biological stability. The elastic material only contains two elements of carbon and hydrogen, so it is non-polar, does not absorb water, and has no groups that can be hydrolyzed and degraded. The material can be chemically crosslinked when it is heated, without requirements for any other substances such as catalysts or without releasing any small molecule substances.

Since the thermally crosslinkable material can overcome the deficiencies of polyurethane, silica gel materials and SIBS materials in long-term implantation in the human body (easy to degrade and calcify), it can be applied for various medical devices for long-term implantation in the human body, especially for medical devices implantable in the human body that are under long-term stress or need to maintain their shapes permanently (such as heart valves, intraocular lenses, glaucoma drainage tubes, lacrimal canalicular plugs, medical occluders, vertebral discs, articular meniscus, artificial ligaments, artificial menisci, vascular grafts, cardiac pacemaker headers and lead insulators, etc.).

After the polymer capable of forming elastic material by thermal crosslinking is cross-linked, it can be applied in medical devices (including heart valve prostheses and intraocular lenses) for long-term implantation in the human body.

The polymer capable of forming elastic material by thermal crosslinking has the following beneficial effects.

1. The polymer capable of forming elastic material by thermal crosslinking is prepared by anionic polymerization. After it is crosslinked by heating, the resulted elastic material has a narrow molecular weight distribution and does not contain oligomers that are easy to filter out when implanted into the human body, with safer use.

2. The prepared polymer capable of forming elastic material by thermal crosslinking suffers from active anionic polymerization and selective catalytic hydrogenation. The polymer only contains two elements of carbon and hydrogen without halogen, so the polymer will not corrode the equipments or cause defects during processing, for example, during making delicate medical devices such as intraocular lenses.

3. The active anionic polymerization uses cheap n-butyllithium as the initiator, and the polymerization temperature is about 50-90° C. Although selective catalytic hydrogenation is required, such polymer synthesis process makes the polymer relatively inexpensive and easy to scale up.

4. The active anionic polymerization has a more flexible molecular design, in which the selection of monomers, the structure of the polymer, and the controllability of polymerization are more flexible than active cationic polymerization. For example, styrene monomer units for the rubber phase of the polymer can be introduced by random copolymerization of styrene and conjugated diene, which is difficult to achieve by active cationic polymerization.

5. The elastic material obtained by anionic polymerization has excellent mechanical properties and higher tensile strength than SIBS. For example, by introducing styrene for random copolymerization for the rubber phase, the tear resistance/wear resistance/tensile modulus can be improved, and the performance can approach to that of polyurethane, to meet higher performance requirements for specific applications.

6. By introducing styrene monomer for the rubber phase, a higher styrene content can be achieved while maintaining proper softness and rubber elasticity, a higher refractive index and transparency can be obtained, and the glass transition temperature of the rubber phase can be raised to close to or even exceed room temperature. This is especially critical for foldable intraocular lens technology, which provides the intraocular lens with superior optical performance, allows the intraocular lens to be thinner and unfold slowly (approximately 15 seconds) after being folded and implanted into the human body.

7. The rubber phase of the material (i.e., the soft segment) contains conjugated diene. After polymerization, the residual double bonds in the conjugated diene monomer units of the polymer can be saturated through selective hydrogenation, thereby obtaining better stability and other more excellent mechanical properties. Meanwhile, the thermally crosslinking monomer contained in the polymer is not affected by active anionic polymerization and selective catalytic hydrogenation, so the hydrogenated polymer can be chemically crosslinked by heating (at about 240° C. for about 20 minutes).

8. The rubber phase of the material can be chemically grafted and modified to obtain more performances and achieve more effects, which is impossible for SIBS materials (polyisobutylene does not have the activity for chemical graft modification).

9. The elastic material has been freed of various impurities (including catalysts, solvents and other impurities) through the purification process before cross-linking, and thus can be chemically cross-linked when heated, without requirements for any other substances such as catalysts and without releasing any small molecule substances. Chemical crosslinking can improve the dimensional stability of the material under high temperature and stress.

10. The elastic material is completely non-polar, does not absorb water, and has no groups that can be hydrolyzed and degraded. After selective hydrogenation, there is no unstable double bond in the molecular structure, and thus good high-temperature-oxidation resistance, biological stability and biocompatibility can be achieved.

11. The thermally crosslinkable polymer elastic material can be used to prepare a new generation of intraocular lenses, overcoming the shortcomings of the existing silica gel and acrylate-based intraocular lenses.

12. After cross-linking, the thermally crosslinkable polymer elastic material can be disinfected by ultraviolet light, gamma ray, electron beam, ethylene oxide, etc, with various disinfection options, and thus it is suitable to be applied for medical devices for long-term implantation.

A preparation method for a polymer capable of forming elastic material by thermal crosslinking and synthesized by anionic polymerization, includes steps of:
(1) performing anionic polymerization of vinyl aromatic hydrocarbon, conjugated diene and thermally crosslinking monomer in solution in presence of anionic polymerization initiator, in an inert gas atmosphere and at a polymerization temperature of −30-150° C.;
wherein amounts of monomer units involved in the polymerization are: a weight percentage of the vinyl aromatic hydrocarbon is 0.01-80%, a weight percentage of the conjugated diene is 20-99.99%, and a weight percentage of the thermally crosslinking monomer is 0.01-30%; and
the thermally crosslinking monomer has a chemical structural formula of:

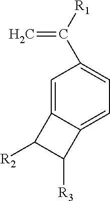

wherein, R₁, R₂ and R₃ are each hydrogen or C₁-C₁₀ alkyl group, and are independent of each other; and
(2) performing catalytic hydrogenation after the polymerization to obtain the polymer capable of forming elastic material by thermal crosslinking.

All elastic materials in the present disclosure can be prepared by this method. Step (1) is an anionic polymerization reaction, and the respective monomers are all monomers that can participate in the anionic polymerization reaction.

The following further provides several options. They are not used as additional limitations on the above-mentioned subject matter, but are only further additions or preferences. Without technical or logical contradiction, the options can be combined with the above-mentioned subject matter independently or in combination.

Vinyl aromatic hydrocarbon, conjugated diene and thermally crosslinking monomer undergo anionic polymerization in the presence of the initiator, and the polymerization product is subjected to selective hydrogenation treatment to convert the unsaturated double bonds of the conjugated diene monomer units into saturated carbon-carbon bonds. The selective hydrogenation product is subjected to purification steps such as removing catalysts and solvents, and then vacuum-dried to obtain a pure polymer capable of forming elastic material by thermal crosslinking. The elastic material can be chemically cross-linked after heating, to obtain a thermosetting elastic material, which can be used to make medical devices or parts thereof, especially medical device products that would be subjected to stress or need to maintain dimensional stability.

The four-membered ring of the thermally crosslinking monomer unit used herein neither prevents the polymerization reaction nor is destroyed by the polymerization process, and the four-membered ring of the thermally crosslinking monomer unit neither affects the hydrogenation reaction, nor is destroyed by the hydrogenation process; the washing process, especially the peroxide treatment, does not destroy the four-membered ring of the thermally crosslinking monomer unit. These factors attributes to the successful synthesis of the target polymer, so as to achieve thermal crosslinking to obtain the desired properties (creep resistance, fatigue resistance, etc.).

In the processes of anionic polymerization, catalytic hydrogenation, washing and purification, etc., the four-membered ring structure of the thermally crosslinking monomer is not destroyed. When the elastic material is heated, the four-membered ring structure is opened to form a chemically crosslinked structure to obtain thermosetting elastic material.

During the anionic polymerization, the block structure of the product is controlled by controlling the feeding sequence, that is, the block structure is formed by feeding reactants in sequence. In the process of anionic polymerization, considering factors such as the reaction speed control, product structure, and molecular weight distribution, an appropriate amount of ingredients can be fed at the beginning of the reaction process, and the ingredients can be continuously and slowly fed during the reaction process.

Optionally, the anionic polymerization initiator is an organolithium compound having the general formula of RLiₙ, wherein R is an aliphatic hydrocarbon group, an alicyclic hydrocarbon group, an aromatic hydrocarbon group or an alkyl-substituted aromatic hydrocarbon group, n is an integer in the range from 1 to 4.

The aliphatic hydrocarbon group is an open chain, that is, a hydrocarbon group that does not contain a ring structure, while the alicyclic hydrocarbon group contains ring structure(s). The substituted alkyl group in the alkyl-substituted aromatic hydrocarbon group is an alkyl group containing 1 to 10 carbon atoms.

Optionally, the anionic polymerization initiator is an organolithium compound having the general formula of RLiₙ, wherein R is an aliphatic hydrocarbon group, an alicyclic hydrocarbon group, an aromatic hydrocarbon group or an alkyl-substituted aromatic hydrocarbon group containing 1 to 10 carbon atoms, n is an integer in the range from 1 to 4, and the substituted alkyl group in the alkyl-substituted aromatic hydrocarbon group is an alkyl group containing 1 to 5 carbon atoms.

Optionally, the anionic polymerization initiator is an organolithium compound having the general formula of RLiₙ, wherein R is an aliphatic hydrocarbon group or an alicyclic hydrocarbon group containing 1 to 5 carbon atoms, and n is an integer in the range from 1 to 4.

Optionally, the initiator is n-butyllithium or sec-butyllithium.

When performing anionic polymerization, the polymerization reaction is carried out in a solvent. Optionally, the solvent is a non-polar saturated aliphatic hydrocarbon or cycloalkane (without ionizable hydrogen atoms).

The solvent needs to meet the requirements for anionic polymerization, for example, can be straight-chain alkanes or cycloalkanes, pentane, hexane, cyclopentane, cyclohexane and the like.

Optionally, the polymerization temperature of the anionic polymerization is in the range from 30° C. to 90° C.; the polymerization time is in the range from 5 min to 5 h.

Optionally, the polymerization temperature of the anionic polymerization is in the range from 50° C. to 70° C.; the polymerization time is in the range from 0.5 h to 2 h.

Optionally, the polymerization environment and the polymerization solvent are pretreated, specifically,
in an anhydrous and anaerobic inert atmosphere, the polymerization solvent that has been dehydrated and deoxygenated by reflux in calcium hydride for 6 to 24 hours is added to the polymerization container, and then alkyl lithium is added to remove impurities.

Optionally, in step (1), a structure regulator is used to control the microstructure of the conjugated diene or vinyl content.

Controlling the microstructure of the conjugated diene by using the structure regulator means controlling the addition mode of the conjugated diene through the structure regulator. Taking 1,3-butadiene as an example, the microstructure of conjugated diene can be controlled by controlling the 1,4-Addition and 1,2-addition ratio of the polymer.

Optionally, the structure regulator is an ether compound. Further preferably, the structure regulator is diethyl ether or tetrahydrofuran.

Optionally, a coupling agent is used during the polymerization in step (1).

Optionally, the coupling agent is an alkoxysilane, the general structural formula thereof is Rₓ—Si—(OR′)_{y}, where the subscript x is 0 or 1, x+y=4, and R and R′ are respectively two independent alkyl groups.

The coupling agent is tetraalkoxysilane or trialkoxysilane. Tetraalkoxysilane includes tetramethoxysilane, tetraethoxysilane, tetrabutoxysilane, tetrakis(2-ethylhexoxy)silane. Trialkoxysilane includes methyltrimethoxysilane, methyltriethoxysilane, isobutylalkylmethoxysilane and phenyltrimethoxysilane. More preferably, the coupling agent is tetramethoxysilane or methyltrimethoxysilane.

Optionally, the coupling agent is chlorosilane. More preferably, the coupling agent is silicon tetrachloride or methyltrichlorosilane.

The purpose of catalytic hydrogenation in step (2) is to hydrogenate carbon-carbon double bonds into saturated carbon-carbon single bonds. Optionally, the specific process of catalytic hydrogenation in step (2) is:
in the presence of the catalyst, the double bonds of the conjugated diene monomer units of the polymer are converted into saturated carbon-carbon bonds, and the degree of catalytic hydrogenation is greater than 80% (that is, more than 80% of the double bonds are hydrogenated); meanwhile, vinyl aromatic hydrocarbons and thermally crosslinking monomer structures are remained.

More preferably, the degree of catalytic hydrogenation is greater than 90%. More preferably, the degree of catalytic hydrogenation is greater than 95%.

Selective hydrogenation catalysts have been reported in the literatures and are widely used in industry. The catalyst system generally consists of two parts: iron group metals (such as nickel, cobalt) and appropriate reducing agents. Catalysts can be formulated using appropriate solvents at temperatures ranging from 20-80° C. Other catalyst systems include titanium-based catalysts, such as titanocene. Generally, nickel-based and cobalt-based catalysts have high catalytic activity and are suitable for selectively catalyzing all conjugated diene monomer units; while titanium-based catalysts have low activity and are generally used to selectively catalyze butadiene monomer units and the like.

Herein, depending on the structure of the polymer, common catalysts in the prior art can be used for hydrogenation catalysis, without special limitations.

Optionally, the catalyst includes an iron group metal and a reducing agent coordinated therewith. When in use, the catalyst is dissolved in a solvent at 20-80° C. Herein, the catalyst is dissolved in the solvent and then added to the reaction system. The solvent can be the same as that of the anionic polymerization reaction, or be different, provided that it will not cause adverse effects on the product.

Optionally, the catalyst includes an iron group metal and a reducing agent coordinated therewith that are dissolved in a solvent at 20-80° C.

Optionally, the catalyst includes nickel 2-ethylhexanoate and triisobutylaluminum dissolved in cyclohexane.

Optionally, the catalyst is a titanium-based catalyst. More preferably, the catalyst is titanocene.

The specific process of the purification is:
a hydrogen peroxide solution is used to oxidize and clean the catalytically hydrogenated polymer solution, and a citric acid aqueous solution is used to extract and remove the catalyst component; then the polymer phase is washed to remove the solvent to obtain the purified polymer capable of forming elastic material by thermal crosslinking.

The preparation method of the polymer capable of forming elastic material by thermal crosslinking generally includes the following steps:
1) adding the dehydrated and deoxygenated solvent of cyclohexane into the reaction vessel at 50-90° C. in an anhydrous and anaerobic inert atmosphere, and using alkyl lithium for impurity removal;
2) adding the initiator alkyl lithium quantitatively, then adding the monomers including the thermally crosslinking monomer (vinyl aromatic hydrocarbon, conjugated diene, thermally crosslinking monomer) in sequence for the polymerization reaction of segments, and finally adding the chain terminator (usually alcohols) to end the polymerization reaction;
3) performing selective catalytic hydrogenation of the polymerization product in solution, using nickel-based, cobalt-based or titanium-based catalyst, at 50-90° C. and 1.6-3.0 MPa pressure, wherein addition reaction of hydrogens and unsaturated double bonds of conjugated diene monomer units occurs, without affecting the molecular structure of vinyl aromatic hydrocarbon and thermally crosslinking monomer units; and
4) after selective catalytic hydrogenation, obtaining the thermally crosslinkable elastic material by subjecting the polymer to a series of purification steps to remove the catalyst and solvent and then vacuum-drying to constant weight. The obtained elastic material can be directly cross-linked at high temperature, to transform the polymer capable of forming elastic material by thermal crosslinking into a thermosetting elastic material.

An elastic material is formed by the polymer capable of forming elastic material through thermal crosslinking.

An interventional device, in which the elastic material is applied, the interventional device includes intraocular lenses, prosthetic valves, glaucoma drainage tubes, lacrimal canalicular plugs, medical occluders, artificial vertebral discs, artificial joints, artificial ligaments, artificial menisci, vascular grafts, cardiac pacemakers (pacemaker headers) and lead insulators, etc.

A polymer for heart valve prosthesis that is capable of forming elastic material by thermal crosslinking is provided, and the elastic material includes polymer A as hard segment and polymer B as soft segment which are polymerized to form a copolymer, with a chemical formula of:

(Aₘ)ᵢ(Bₙ)ⱼ(A_{f})ₖ; or (Aₘ-Bₙ)ₚX(Bₙ−A_{f})_{q};
wherein compositions of polymers A at two ends of the polymer B are independent of each other;
the polymer A is a polymer formed by polymerization of at least one of vinyl aromatic hydrocarbon and thermally crosslinking monomer, or a polymer formed by copolymerization of at least one of vinyl aromatic hydrocarbon and thermally crosslinking monomer with conjugated diene;
the polymer B is a conjugated diene polymer, or a polymer formed by copolymerization of at least one of vinyl aromatic hydrocarbon and thermally crosslinking monomer with conjugated diene;
at least one of the polymer A and the polymer B contains the thermally crosslinking monomer;
X represents a residual group of a coupling agent after coupling reaction;
subscripts m and f each represents a number of comonomer units of the polymer A, subscript n represents a number of comonomer units of the polymer B, and m, n, f are all integers greater than or equal to 1 and independent of each other;
subscripts i and k each represents a number of blocks of the polymer A, subscript j represents a number of blocks of the polymer B, both i and k are integers greater than or equal to 0, j is an integer greater than or equal to 1, and i, j, k are independent of each other;
subscripts p and q each represents a number of blocks formed by polymerization of the polymer A and the polymer B, and both p and q are integers greater than or equal to 0 and are independent of each other;
the thermally crosslinking monomer has a chemical structural formula of:

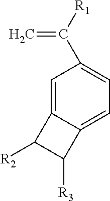

wherein, R₁, R₂ and R₃ are each hydrogen or C₁-C₁₀ alkyl group, and are independent of each other. Optionally, at least one of the polymer A and the polymer B contains the vinyl aromatic hydrocarbon.

Optionally, the polymer A is the polymer formed by polymerization of at least one of vinyl aromatic hydrocarbon and thermally crosslinking monomer, or the polymer formed by copolymerization of at least one of vinyl aromatic hydrocarbon and thermally crosslinking monomer with conjugated diene;
the polymer B is the conjugated diene polymer, or the polymer formed by copolymerization of at least one of vinyl aromatic hydrocarbon and thermally crosslinking monomer with conjugated diene; and
when the polymer A is the polymer formed by polymerization of vinyl aromatic hydrocarbon and thermally crosslinking monomer, the polymer B is the polymer formed by copolymerization of at least one of vinyl aromatic hydrocarbon and thermally crosslinking monomer with conjugated diene.

Optionally, the polymer A is a copolymer of the vinyl aromatic hydrocarbon and the thermally crosslinking monomer; and

the polymer B is a copolymer of the vinyl aromatic hydrocarbon, the conjugated diene and the thermally crosslinking monomer.

Optionally, the polymer A is a copolymer of the vinyl aromatic hydrocarbon and the thermally crosslinking monomer; and
the polymer B is a copolymer of the vinyl aromatic hydrocarbon and the conjugated diene.

Optionally, the polymer A is a copolymer of the vinyl aromatic hydrocarbon and the thermally crosslinking monomer; and
the polymer B is a copolymer of the conjugated diene and the thermally crosslinking monomer.

Optionally, the polymer A is a copolymer of the vinyl aromatic hydrocarbon, the conjugated diene and the thermally crosslinking monomer; and
the polymer B is a copolymer of the vinyl aromatic hydrocarbon, the conjugated diene and the thermally crosslinking monomer.

Optionally, the polymer A is a copolymer of the vinyl aromatic hydrocarbon, the conjugated diene and the thermally crosslinking monomer; and
the polymer B is a copolymer of the vinyl aromatic hydrocarbon and the conjugated diene.

Optionally, the polymer A is a copolymer of the vinyl aromatic hydrocarbon, the conjugated diene and the thermally crosslinking monomer; and
the polymer B is a copolymer of the conjugated diene and the thermally crosslinking monomer.

Optionally, the polymer A is a polymer of the vinyl aromatic hydrocarbon; and
the polymer B is a copolymer of the vinyl aromatic hydrocarbon, the conjugated diene and the thermally crosslinking monomer.

Optionally, the polymer A is a polymer of the vinyl aromatic hydrocarbon; and
the polymer B is a copolymer of the conjugated diene and the thermally crosslinking monomer.

Optionally, i, j, and k are each 1, and m, n, and f are each in the range of 10-100.

Optionally, the content of the vinyl aromatic hydrocarbon in the polymer A is 80-100%.

Optionally, the content of the vinyl aromatic hydrocarbon in the polymer A is 95-100%.

Optionally, the content of the vinyl aromatic hydrocarbon in the polymer B is 0-60%.

Optionally, the content of the vinyl aromatic hydrocarbon in the polymer B is 20-40%.

Optionally, the content of the conjugated diene in the polymer A is 0-20%.

Optionally, the content of the conjugated diene in the polymer A is 0-10%.

Optionally, the content of the conjugated diene in the polymer B is 40-100%.

Optionally, the content of the conjugated diene in the polymer B is 60-90%.

Optionally, the content of the thermally crosslinking monomer in the polymer A is 0.1-5%.

Optionally, the content of the thermally crosslinking monomer in the polymer A is 1-2%.

Optionally, the content of the thermally crosslinking monomer in the polymer B is 0.1-5%.

Optionally, the content of the thermally crosslinking monomer in the polymer B is 1-2%.

Optionally, the polymer A is a copolymer of the vinyl aromatic hydrocarbon and the thermally crosslinking monomer, and wherein a content of the vinyl aromatic hydrocarbon in the polymer A is 95-100%, with the remaining being the thermally crosslinking monomer.

Optionally, the polymer A is formed by polymerization of the vinyl aromatic hydrocarbon, the conjugated diene and thermally crosslinking monomer, and wherein a content of the vinyl aromatic hydrocarbon in the polymer A is 80-100%, a content of the conjugated diene in the polymer A is not more than 20%, with the remaining being the thermally crosslinking monomer.

Optionally, the polymer B is formed by polymerization of the vinyl aromatic hydrocarbon and the conjugated diene, and wherein a content of the vinyl aromatic hydrocarbon in the polymer B is 0-60%, with the remaining being the conjugated diene.

Optionally, the polymer B is formed by polymerization of the conjugated diene and the thermally crosslinking monomer, and wherein a content of the conjugated diene in the polymer B is 90-100%, with the remaining being the thermally crosslinking monomer.

Optionally, the polymer B is formed by polymerization of the vinyl aromatic hydrocarbon, the conjugated diene and the thermally crosslinking monomer, and wherein a content of the vinyl aromatic hydrocarbon in the polymer B is 0-60%, a content of the conjugated diene in the polymer B is 40-100%, with the remaining being the thermally crosslinking monomer.

Optionally, the polymer B is formed by polymerization of the vinyl aromatic hydrocarbon, the conjugated diene and the thermally crosslinking monomer, and wherein a content of the vinyl aromatic hydrocarbon in the polymer B is 20-50%, a content of thermally crosslinking monomer is not more than 5%, with the remaining being the conjugated diene.

Optionally, the vinyl aromatic hydrocarbon is styrene, the conjugated diene is at least one of isoprene and 1,3-butadiene, and the thermally crosslinking monomer is 4-vinylbenzocyclobutene.

Optionally, the vinyl aromatic hydrocarbon is styrene, the conjugated diene is 1,3-butadiene, and the thermally crosslinking monomer is 4-vinylbenzocyclobutene.

Optionally, the molecular weight of the polymer that is capable of forming elastic material by thermal crosslinking is 20000-1000000.

Optionally, the molecular weight of the polymer that is capable of forming elastic material by thermal crosslinking is 20000-500000.

Optionally, the molecular weight of the polymer that is capable of forming elastic material by thermal crosslinking is 50000-150000.

Herein, the molecular weight refers to the relative number average molecular weight measured by GPC and calibrated with polystyrene standard.

Optionally, the molecular weight distribution of the polymer capable of forming elastic material by thermal crosslinking is 1.0-1.3.

Optionally, the molecular weight distribution of the polymer capable of forming elastic material by thermal crosslinking is 1.0-1.1.

Optionally, a tensile strength of the polymer that is capable of forming elastic material by thermal crosslinking is greater than 10 MPa, and the breaking elongation is greater than 300%.

Optionally, a tensile strength of the polymer that is capable of forming elastic material by thermal crosslinking is greater than 15 MPa, and the breaking elongation is greater than 500%.

Optionally, a tensile strength of the polymer that is capable of forming elastic material by thermal crosslinking is greater than 20 MPa, and the breaking elongation is greater than 500%.

A heart valve prosthesis is formed by the polymer capable of forming elastic material by thermal crosslinking by thermally crosslinking.

The polymer capable of forming elastic material by thermal crosslinking is heated and cross-linked to form a solid elastic material, and the solid elastic material is further processed, such as being cut and sewn among others, to form leaflets of a heart valve prosthesis.

Before preparing the heart valve prosthesis, it is necessary to transform the polymer capable of forming elastic material by thermal crosslinking into a thermosetting elastic material film at high temperature.

The heart valve prosthesis can be implanted into the human body through a catheter.

The heart valve prosthesis can be implanted through thoracotomy or minimally invasive replacement through a small incision.

The heart valve prosthesis includes an aortic valve, a pulmonary valve, a mitral valve, a tricuspid valve or a venous valve implantable through thoracotomy or minimally invasive replacement through a small incision.

The heart valve prosthesis is an aortic valve, a pulmonary valve, a mitral valve or a tricuspid valve.

The heart valve prosthesis is an aortic valve and can be implanted by transthoracic aortic valve implantation or balloon dilatation of aortic valve.

The thickness of the heart valve prosthesis is 0.02-0.40 mm.

The thickness of the heart valve prosthesis is 0.05-0.40 mm.

The thickness of the heart valve prosthesis is 0.10-0.15 mm.

The thickness of the heart valve prosthesis is 0.08-0.15 mm.

Herein, the thickness of the heart valve prosthesis refers to the thickness of the leaflet.

The heart valve prosthesis prepared by cross-linking the polymer capable of forming elastic material by thermal crosslinking can be compressed and then introduced through a catheter, and deployed at a suitable site of the heart by self-expanding or balloon-expanding.

The heart valve prosthesis is prepared by the polymer capable of forming elastic material by thermal crosslinking according to the present invention. The polymer capable of forming elastic material by thermal crosslinking provided by the invention is synthesized by active anionic polymerization, and does not contain small molecular additives or residual components. The elastic material has no unstable double bonds in the molecular structure after selective catalytic hydrogenation, and thus has excellent high-temperature-oxidation resistance and biological stability. The elastic material only contains two elements of carbon and hydrogen, so it is non-polar, does not absorb water, and has no groups that can be hydrolyzed and degraded. The material can be chemically crosslinked when it is heated, without requirements for any other substances such as catalysts and without releasing any small molecule substances. After being cross-linked by heat, the material can be applied for various medical devices implantable in the human body that are under long-term stress or need to maintain their shapes permanently, including heart valve prostheses.

The polymer for preparing heart valve prosthesis that is capable of forming elastic material by thermal crosslinking according to the present invention has excellent biological stability, anti-coagulation performance, anti-calcification performance and mechanical strength, and has creep resistance after cross-linking. Therefore, the heart valve prostheses prepared from the materials can overcome the shortcomings of other heart valve prostheses (such as mechanical valves and biological valves).

The polymer material according to the present invention can be mass-produced, and its performance and quality can be stably controlled, thereby greatly reducing the cost of heart valve products. The polymer material according to the present invention has various mechanical properties, which can be designed in molecular structure and chemical composition to achieve the performances required for heart valve products (including soft and foldable properties to meet the requirements for minimally invasive surgery such as transcatheter intervention). The polymer material according to the present invention can be processed through different methods to obtain heart valves of different sizes and shapes, while biological valves can only be cut and sewed and cannot be reprocessed to change the thickness and shape in most situations. The polymer material according to the present invention does not carry diseases of animal origin.

### BRIEF DESCRIPTION OF DRAWINGS

FIG. 1a is a comparison of ¹H-NMR of the elastic material obtained in Example 1 before and after hydrogenation;

FIG. 1B is a comparison of GPC of the elastic material obtained in Example 1 before and after hydrogenation;

FIG. 1c is a DSC test result of the elastic material obtained in Example 1 before and after hydrogenation;

FIG. 2a is a comparison of ¹H-NMR of the elastic material obtained in Example 2 before and after hydrogenation;

FIG. 2b is a comparison of GPC of the elastic material obtained in Example 2 before and after hydrogenation;

FIG. 2c is a DSC test result of the elastic material obtained in Example 2 before and after hydrogenation;

FIG. 3a is a comparison of ¹H-NMR of the elastic material obtained in Example 3 before and after hydrogenation;

FIG. 3b is a comparison of GPC of the elastic material obtained in Example 3 before and after hydrogenation;

FIG. 4a is a comparison of ¹H-NMR of the elastic material obtained in Example 4 before and after hydrogenation;

FIG. 4b is a comparison of GPC of the elastic material obtained in Example 4 before and after hydrogenation;

FIG. 5a is a comparison of ¹H-NMR of the elastic material obtained in Example 5 before and after hydrogenation;

FIG. 5b is a comparison of GPC of the elastic material obtained in Example 5 before and after hydrogenation;

FIG. 6a is a GPC graph of the elastic material obtained in Example 9 before hydrogenation;

FIG. 6b is a ¹H-NMR spectrum of the elastic material obtained in Example 9 before hydrogenation;

FIG. 7a is a GPC graph of the elastic material obtained in Example 10 before hydrogenation;

FIG. 7b is a ¹H-NMR spectrum of the elastic material obtained in Example 10 before hydrogenation;

FIG. 8a is a GPC graph of the elastic material obtained in Example 11 before hydrogenation;

FIG. 8b is a ¹H-NMR spectrum of the elastic material obtained in Example 11 before hydrogenation;

FIG. 9a is a GPC graph of the elastic material obtained in Example 12 before hydrogenation;

FIG. 9b is a ¹H-NMR spectrum of the elastic material obtained in Example 12 before hydrogenation;

FIG. 10a is a GPC graph of the elastic material obtained in Example 13 before hydrogenation;

FIG. 10b is a ¹H-NMR spectrum of the elastic material obtained in Example 13 before hydrogenation;

FIG. 11a is a GPC graph of the elastic material obtained in Example 14 before hydrogenation;

FIG. 11b is a ¹H-NMR spectrum of the elastic material obtained in Example 14 before hydrogenation;

FIG. 12a is a GPC graph of the elastic material obtained in Example 15 before hydrogenation;

FIG. 12b is a ¹H-NMR spectrum of the elastic material obtained in Example 15 before hydrogenation;

FIG. 13 is an infrared spectrum of the elastic material obtained in Example 3 after selective hydrogenation;

FIG. 14 shows the results of the platelet adhesiveness test of elastic materials that can be used to prepare heart valve prostheses;

FIG. 15 shows the results of the blood adhesiveness test of elastic materials that can be used to prepare heart valve prostheses;

FIG. 16 shows the results of blood coagulation four items of elastic materials that can be used to prepare heart valve prostheses; wherein, A shows a PT test result; B shows an APTT test result; C shows a TT test result; D shows a FIB test result;

FIG. 17 shows the results of anti-calcification performance detection of elastic materials that can be used to prepare heart valve prostheses; and

FIG. 18 shows the results of the seam strength under the simulation test of the polymer elastic materials of the present invention and the biological valve material.

### DESCRIPTION OF EXAMPLES

The technical solutions of the examples of the invention will be clearly and fully described in the following with reference to the drawings of the examples of the invention. Obviously, the described examples are only some of the examples of the invention, not all of them. Based on the examples in the present disclosure, all other examples obtained by the skilled person in the art without creative efforts fall into the scope of protection of the invention.

Unless otherwise defined, all technical and scientific terms used herein have the same meaning as commonly understood by the skilled person in the art. The terms used in the specification are only for the purpose of describing specific examples, and are not intended to limit the invention.

In the following examples, the solvents were all pretreated. The pretreatment method is as follows: adding the dehydrated and deoxygenated solvent of cyclohexane into the reaction vessel at 50-90° C. in an anhydrous and anaerobic inert atmosphere, and using alkyl lithium for impurity removal. The pretreatment is to remove impurities that may exist in the solvents, and desired impurity removal effects can be achieved in the range from 50-90° C., which will not be described in detail in the examples.

The molecular structures shown in the examples are only for illustration. In the examples, m and n represent the number of comonomer units in the respective block, wherein each block is a random copolymer, and the proportion of the comonomer units in the block is determined according to the feed ratio.

### Example 1 (Sample Code: RBHX-001)

A thermally crosslinkable random terpolymer elastic material: a product of poly(butadiene-co-styrene-co-4VBCB) (4VBCB is 4-vinylbenzocyclobutene) after selective hydrogenation has the following molecular structure:

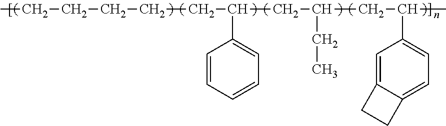

Its preparation method is as follows:

(1) Polymerization process: a styrene/4VBCB mixture (the weight of 4VBCB is 2% of the weight of the mixture) was prepared in advance; 500 mL solvent of cyclohexane (water content is 10 ppm) was added into the polymerization vessel, and heated up to 65° C.; 3.3 mL of styrene/4VBCB mixture and 2 g of butadiene were added therein at the same time, then 0.30 mL of n-butyllithium (1.6 M n-hexane solution) was added therein; then 29.7 mL of styrene/4VBCB mixture was added therein, and 18 g of butadiene was then added dropwise over 25 minutes; after the butadiene was added dropwise, the reaction was continued for 20 minutes, and isopropanol was added therein to terminate the polymerization reaction.

(2) Selective catalytic hydrogenation process: 0.83 g of nickel 2-ethylhexanoate was added into a single-necked flask, and dissolved with 25 mL of cyclohexane, then 6.2 mL of triisobutylaluminum (1.1 M toluene solution) was slowly dropped therein and mixed well, which mixture is the catalyst for selective hydrogenation; the polymer solution after polymerization was transferred to a hydrogenated vessel at 70° C., and the prepared catalyst was added therein, wherein the pressure was boosted to 1.8 MPa with hydrogen while fully stirring, and continuously maintained with hydrogen until hydrogenation is completed.

(3) Hydrogenated polymer washing process: the hydrogenated polymer solution was transferred to a washing vessel at 70° C., and 30 mL of hydrogen peroxide (30%) was added therein and mixed for 30 min; 3% citric acid solution (1 L) was added therein and mixed for 1 hour and then the citric acid solution was separated; 1 L of citric acid solution was continuously added therein for extraction, and then the citric acid solution was separated; the polymer solution was washed with deionized water until it is neutral; the washed polymer was precipitated in isopropanol and vacuum dried to constant weight, thereby obtaining the final hydrogenated product, i.e., the thermally crosslinkable elastic material.

(4) Thermal cross-linking reaction: the elastic material was molded at 240° C. for 20 minutes, and the obtained material was no longer dissolved in toluene (only swelling occurred), indicating that a cross-linking reaction had occurred.

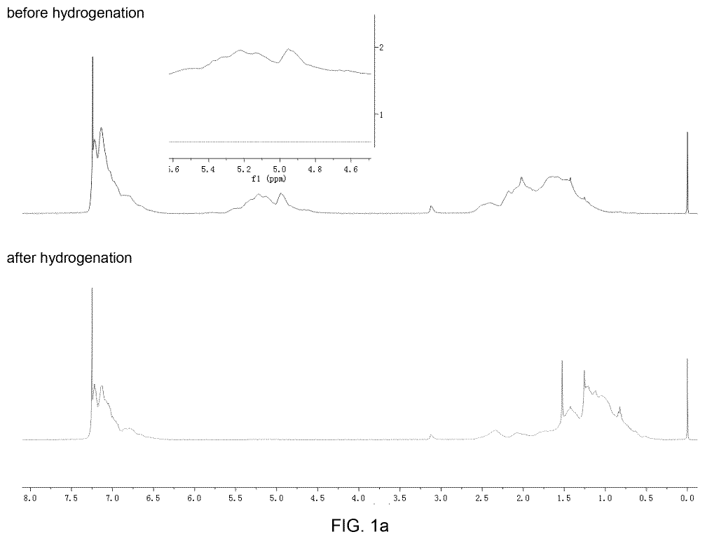

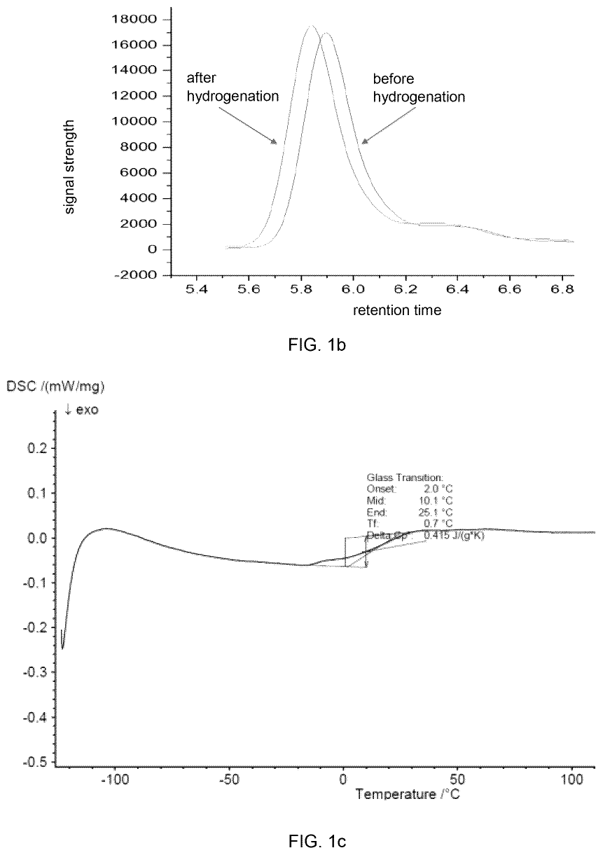

The ¹H-NMR spectra of the material before and after hydrogenation are shown in FIG. 1a, which shows that the residual double bonds (about 4.5-5.8 ppm) of the butadiene monomer unit after selective catalytic hydrogenation have been completely saturated, and the degree of hydrogenation is 100%, while the benzocyclobutene group of the thermally crosslinking monomer unit still exists (about 3.1 ppm). The GPC spectra of the material before and after hydrogenation are shown in FIG. 1b, which shows that the molecular weight distribution before and after hydrogenation remains substantially unchanged.

DSC test results of the material after hydrogenation and crosslinking are shown in FIG. 1c, which shows that the glass transition temperature of the material is ˜10° C. Since the glass transition temperature of the cross-linked material is close to room temperature, the cross-linked sample, which had been folded, restored to its original shape after about 15 s, rather than restoring to its original shape immediately and elastically.

### Example 2 (Sample Code: RIHX-003)

A thermally crosslinkable random terpolymer elastic material: a product of poly(isoprene-co-styrene-co-4VBCB) (4VBCB is 4-vinylbenzocyclobutene) after selective hydrogenation has the following molecular structure:

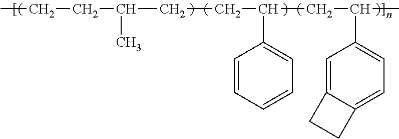

Its preparation method is as follows:

(1) Polymerization process: a styrene/4VBCB mixture (the weight of 4VBCB is 2% of the weight of the mixture) was prepared in advance; 500 mL solvent of cyclohexane (water content is 10 ppm) was added into the polymerization vessel, and heated up to 65° C.; 3.3 mL of styrene/4VBCB mixture and 3 mL of isoprene were added therein at the same time, then 0.30 mL of n-butyllithium (1.6 M n-hexane solution) was added therein; then 29.7 mL of styrene/4VBCB mixture was added therein, and 27 mL of isoprene was then added dropwise over 25 minutes; after the isoprene was added dropwise, the reaction was continued for 20 minutes, and isopropanol was added therein to terminate the polymerization reaction.

(2) Selective catalytic hydrogenation process: 1.25 g of nickel 2-ethylhexanoate was added into a single-necked flask, and dissolved with 38 mL of cyclohexane, then 9.3 mL of triisobutylaluminum (1.1 M toluene solution) was slowly dropped therein and mixed well, which mixture is the catalyst for selective hydrogenation; the polymer solution after polymerization was transferred to a hydrogenated vessel at 70° C., and the prepared catalyst was added therein, wherein the pressure was boosted to 1.8 MPa with hydrogen while fully stirring, and continuously maintained with hydrogen until hydrogenation is completed.

(3) Hydrogenated polymer washing process: same as the washing process in Example 1.

(4) Thermal cross-linking reaction: the elastic material was molded at 240° C. for 20 minutes, and the obtained material was no longer dissolved in toluene (only swelling occurred), indicating that a cross-linking reaction had occurred.

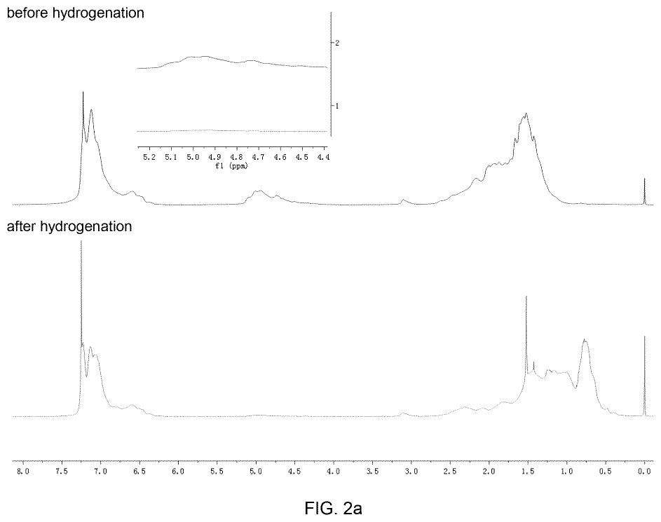

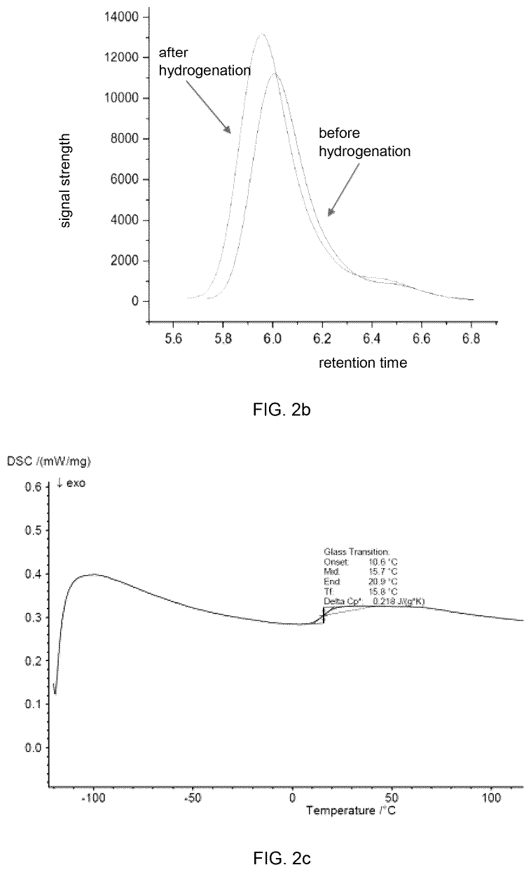

The ¹H-NMR spectra of the elastic material before and after hydrogenation are shown in FIG. 2a, which shows that the residual double bonds (about 4.5-5.2 ppm) of the isoprene monomer unit after selective catalytic hydrogenation are still slightly unsaturated (about 5.1 ppm), and the degree of hydrogenation is 95.8%, while the benzocyclobutene group of the thermally crosslinking monomer unit still exists (about 3.1 ppm). The GPC spectra of the material before and after hydrogenation are shown in FIG. 2b, which shows that the molecular weight distribution before and after hydrogenation remains substantially unchanged.

DSC test results of the material after hydrogenation and crosslinking are shown in FIG. 1c, which shows that the glass transition temperature of the material is ˜15° C. Since the glass transition temperature of the cross-linked material is close to room temperature, the cross-linked sample, which had been folded, restored to its original shape after about 15 s, rather than restoring to its original shape immediately and elastically.

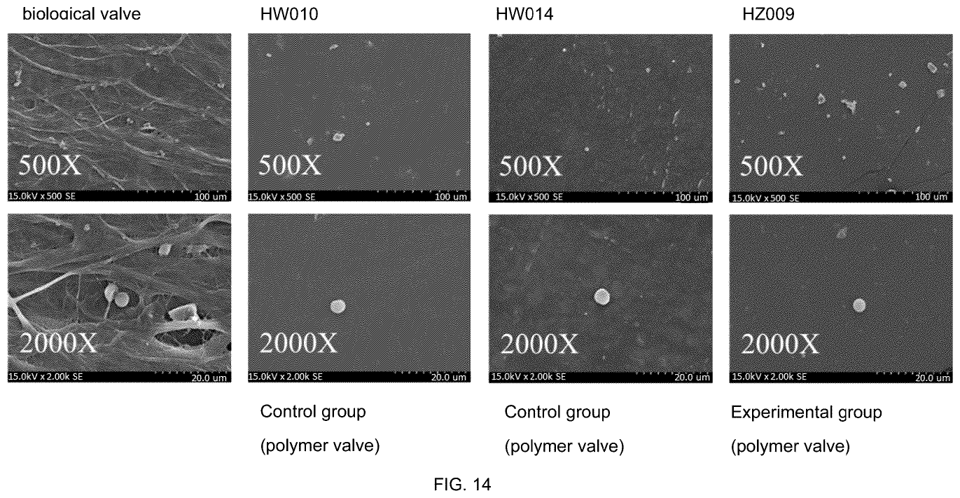

The DSC pictures of the elastic material after selective hydrogenation are shown in FIG. 14. The DSC test method includes: nitrogen protection, temperature change rate of 10° C.; heating up to 150.0° C. for 5 min; then cooling down to −60° C. for 5 min; heating up from −60° C. to 150° C., wherein the glass transition temperature was measured in the second heating process as 13° C.

### Example 3 (Sample Code: RBLX-002)

A thermally crosslinkable triblock polymer elastic material: a product of (styrene-co-4VBCB)-poly(butadiene-co-styrene-co-4VBCB)-poly(styrene-co-4VBCB)(4VBCB is 4-vinylbenzocyclobutene) after selective hydrogenation has the following molecular structure:

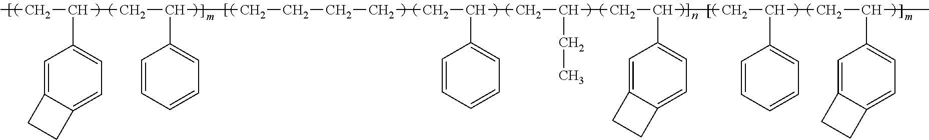

Its preparation method is as follows:

(1) Polymerization process: a styrene/4VBCB mixture (the weight of 4VBCB is 2% of the weight of the mixture) was prepared in advance; 1000 mL solvent of cyclohexane (water content is 10 ppm) was added into the polymerization vessel, and heated up to 70° C.; styrene/4VBCB mixture (14 mL) and 0.55 mL n-butyllithium (1.6 M n-hexane solution) were added in sequence and reacted for 15 minutes; butadiene cyclohexane solution (containing 5.2 g butadiene) and styrene/4VBCB mixture (2.5 mL) were added in sequence, then styrene/4VBCB mixture (20.7 mL) and butadiene cyclohexane solution (containing 11.8 g butadiene) were immediately added in sequence, and then after reaction for 1, 4, and 9 minutes, equal amounts of butadiene cyclohexane solution (containing 11.8 g butadiene) were added therein respectively; after 30 minutes of reaction, styrene/4VBCB mixture (13.75 mL) was added therein for reaction for 30 minutes, and then isopropanol was added therein to terminate the polymerization reaction.

(2) Selective catalytic hydrogenation process: 1.25 g of nickel 2-ethylhexanoate was added into a single-necked flask, and dissolved with 57 mL of cyclohexane, then 9.3 mL of triisobutylaluminum (1.1 M toluene solution) was slowly dropped therein and mixed well, which mixture is the catalyst for selective hydrogenation; the polymer solution after polymerization was transferred to a hydrogenated vessel at 70° C., and the prepared catalyst was added therein, wherein the pressure was boosted to 1.8 MPa with hydrogen while fully stirring, and continuously maintained with hydrogen until hydrogenation is completed.

(3) Hydrogenated polymer washing process: same as the washing process in Example 1.

(4) Thermal cross-linking reaction: the elastic material was molded at 240° C. for 20 minutes, and the obtained material was no longer dissolved in toluene (only swelling occurred), indicating that a cross-linking reaction had occurred.

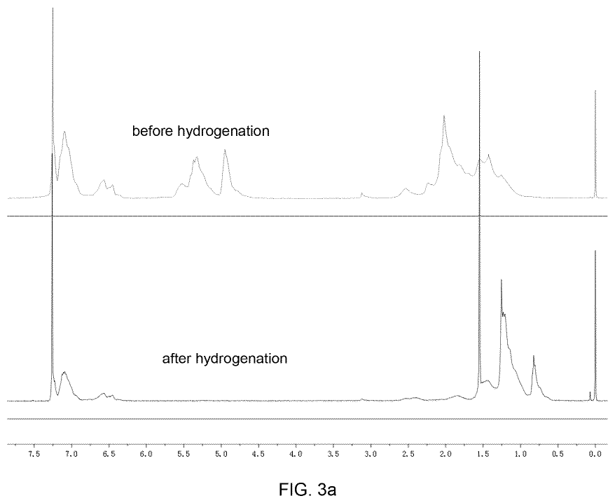

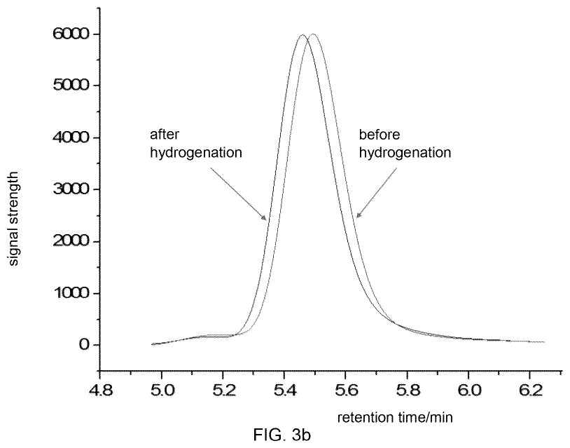

The ¹H-NMR spectra of the elastic material before and after hydrogenation are shown in FIG. 3a, which shows that the residual double bonds (about 4.5-5.8 ppm) of the butadiene monomer unit after selective catalytic hydrogenation have been completely saturated, and the degree of hydrogenation is 100%, while the benzocyclobutene group of the thermally crosslinking monomer unit still exists (about 3.1 ppm). The GPC spectra of the material before and after hydrogenation are shown in FIG. 3b, which shows that the molecular weight distribution before and after hydrogenation remains substantially unchanged.

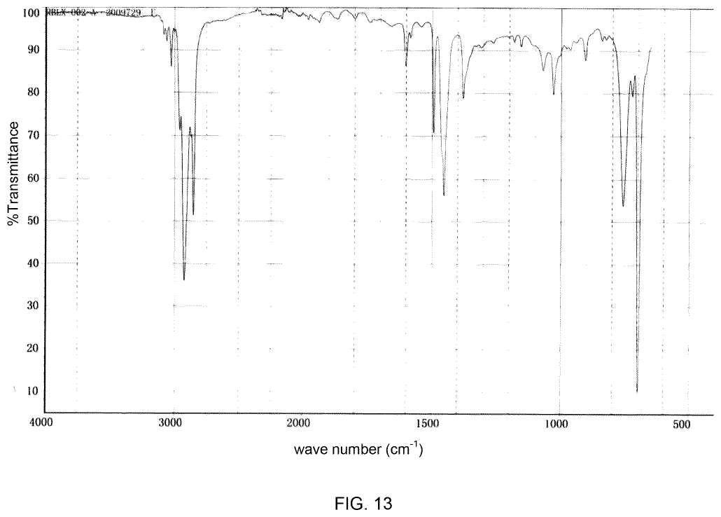

The infrared spectrum of the elastic material after hydrogenation is shown in FIG. 13.

### Example 4 (Sample Code ILX-001)

A thermally crosslinkable triblock polymer elastic material: a product of (styrene-co-4VBCB)-polyisoprene-poly(styrene-co-4VBCB) after selective hydrogenation has the following molecular structure:

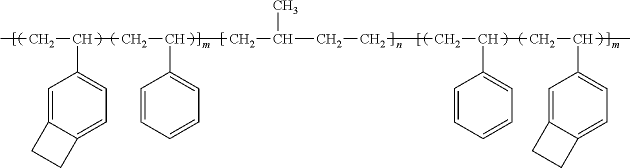 [SMILES: CCC(CC(CC(C)CCCC(CC(C)c1ccc2c(c1)CC2)c1ccccc1)c1ccccc1)c1ccc2c(c1)CC2]

Its preparation method is as follows:

(1) Polymerization process: a styrene/4VBCB mixture (the weight of 4VBCB is 2% of the weight of the mixture) was prepared in advance; 1000 mL solvent of cyclohexane (water content is 10 ppm) was added into the polymerization vessel, and heated up to 70° C.; styrene/4VBCB mixture (16.5 mL) and 0.50 mL n-butyllithium (1.6 M n-hexane solution) were added in sequence and reacted for 15 minutes; 103 mL isoprene was added therein for reaction for 30 minutes; 16.5 mL styrene/4VBCB mixture was added therein for reaction for 30 minutes, and then isopropanol was added therein to terminate the polymerization reaction.

(2) Selective catalytic hydrogenation process: 1.25 g of nickel 2-ethylhexanoate was added into a single-necked flask, and dissolved with 57 mL of cyclohexane, then 9.3 mL of triisobutylaluminum (1.1 M toluene solution) was slowly dropped therein and mixed well, which mixture is the catalyst for selective hydrogenation; the polymer solution after polymerization was transferred to a hydrogenated vessel at 70° C., and the prepared catalyst was added therein, wherein the pressure was boosted to 1.8 MPa with hydrogen while fully stirring, and continuously maintained with hydrogen until hydrogenation is completed.

(3) Hydrogenated polymer washing process: same as the washing process in Example 1.

(4) Thermal cross-linking reaction: the elastic material was molded at 240° C. for 20 minutes, and the obtained material was no longer dissolved in toluene (only swelling occurred), indicating that a cross-linking reaction had occurred.

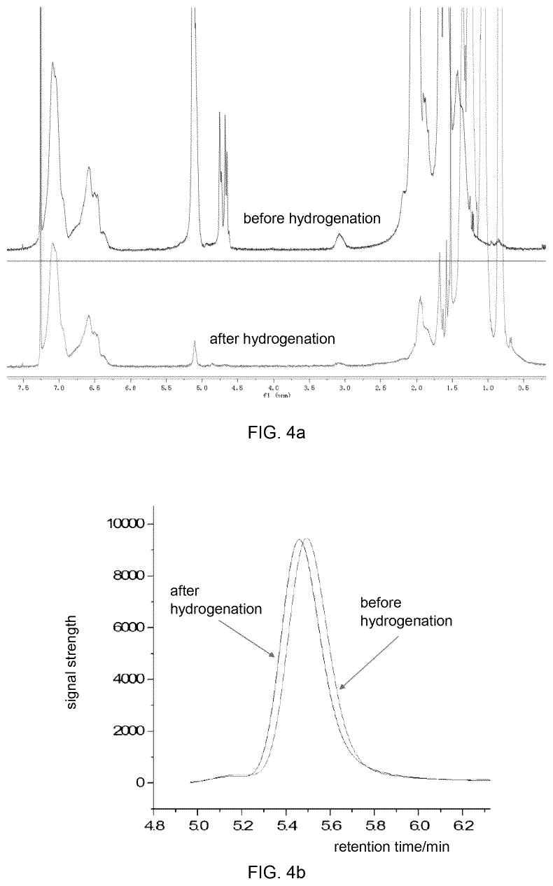

The ¹H-NMR spectra of the elastic material before and after hydrogenation are shown in FIG. 4a, which shows that the residual double bonds (about 4.5-5.2 ppm) of the isoprene monomer unit after selective catalytic hydrogenation are still slightly unsaturated (about 5.1 ppm), and the degree of hydrogenation is 95.2%, while the benzocyclobutene group of the thermally crosslinking monomer unit still exists (about 3.1 ppm). The GPC spectra of the material before and after hydrogenation are shown in FIG. 4b, which shows that the molecular weight distribution before and after hydrogenation remains substantially unchanged.

### Example 5 (Sample Code: RILX-004)

A thermally crosslinkable triblock polymer elastic material: a product of (styrene-co-4VBCB)-poly(isoprene-co-styrene-co-4VBCB)-poly(styrene-co-4VBCB) after selective hydrogenation has the following molecular structure:

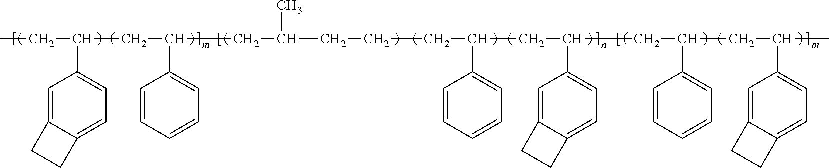 [SMILES: CCC(CC(CC(C)CCCC(CC(CC(CC(C)c1ccc2c(c1)CC2)c1ccccc1)c1ccc2c(c1)CC2)c1ccccc1)c1ccccc1)c1ccc2c(c1)CC2]

Its preparation method is as follows:

(1) Polymerization process: a styrene/4VBCB mixture (the weight of 4VBCB is 2% of the weight of the mixture) was prepared in advance; 1000 mL solvent of cyclohexane (water content is 10 ppm) was added into the polymerization vessel, and heated up to 70° C.; styrene/4VBCB mixture (16.5 mL) and 0.50 mL n-butyllithium (1.6 M n-hexane solution) were added in sequence and reacted for 15 minutes; 7.2 mL of isoprene and 2.3 mL of styrene/4VBCB mixture were added in sequence, then 20.7 mL of styrene/4VBCB mixture and 16.2 mL of isoprene were added in sequence, and then after reaction for 3, 8, and 17 minutes, 16.2 mL of isoprene was added therein respectively; after 30 minutes of reaction, 16.5 mL of styrene/4VBCB mixture was added therein for reaction for 30 minutes, and then isopropanol was added therein to terminate the polymerization reaction.

(2) Selective catalytic hydrogenation process: 1.25 g of nickel 2-ethylhexanoate was added into a single-necked flask, and dissolved with 57 mL of cyclohexane, then 9.3 mL of triisobutylaluminum (1.1 M toluene solution) was slowly dropped therein and mixed well, which mixture is the catalyst for selective hydrogenation; the polymer solution after polymerization was transferred to a hydrogenated vessel at 70° C., and the prepared catalyst was added therein, wherein the pressure was boosted to 1.8 MPa with hydrogen while fully stirring, and continuously maintained with hydrogen until hydrogenation is completed.

(3) Hydrogenated polymer washing process: same as the washing process in Example 1.

(4) Thermal cross-linking reaction: the elastic material was molded at 240° C. for 20 minutes, and the obtained material was no longer dissolved in toluene (only swelling occurred), indicating that a cross-linking reaction had occurred.

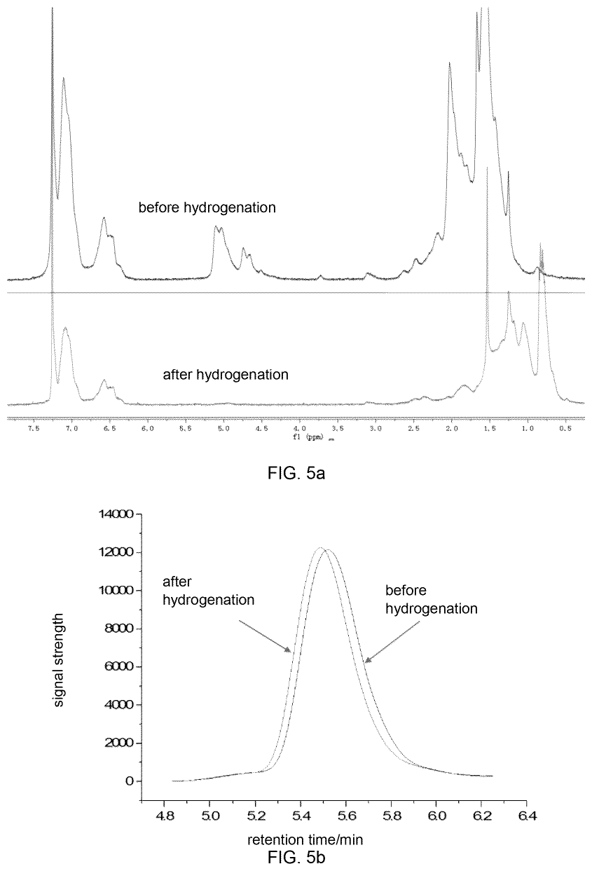

The ¹H-NMR spectra of the elastic material before and after hydrogenation are shown in FIG. 5a, which shows that the residual double bonds (about 4.5-5.2 ppm) of the isoprene monomer unit after selective catalytic hydrogenation basically disappear, and the degree of hydrogenation is close to 100%, while the benzocyclobutene group of the thermally crosslinking monomer unit still exists (about 3.1 ppm). The GPC spectra of the material before and after hydrogenation are shown in FIG. 5b, which shows that the molecular weight distribution before and after hydrogenation remains substantially unchanged.

### Example 6 (Sample Code: BLX-001)

A thermally crosslinkable triblock polymer elastic material: a product of (styrene-co-4VBCB)-polybutadiene-poly(styrene-co-4VBCB) after selective hydrogenation has the following molecular structure:

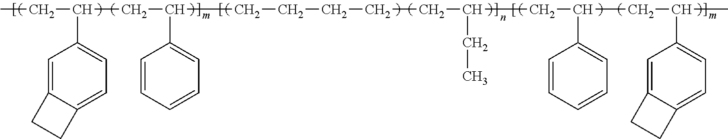 [SMILES: CCC(CCCCCC(CC(CC)c1ccc2c(c1)CC2)c1ccccc1)CC(CC(C)c1ccc2c(c1)CC2)c1ccccc1]

Its preparation method is as follows:

(1) Polymerization process: a styrene/4VBCB mixture (the weight of 4VBCB is 2% of the weight of the mixture) was prepared in advance; 450 mL solvent of cyclohexane (water content is 10 ppm) was added into the polymerization vessel, and heated up to 75° C.; styrene/4VBCB mixture (6.9 mL) and 0.13 mL n-butyllithium (1.6 M n-hexane solution) were added in sequence and reacted for 15 minutes; 39.5 g butadiene was added therein for reaction for 30 minutes; 6.9 mL styrene/4VBCB mixture was added therein for reaction for 20 minutes, and then isopropanol was added therein to terminate the polymerization reaction.

(2) Selective catalytic hydrogenation process: 1.25 g of nickel 2-ethylhexanoate was added into a single-necked flask, and dissolved with 57 mL of cyclohexane, then 9.3 mL of triisobutylaluminum (1.1 M toluene solution) was slowly dropped therein and mixed well, which mixture is the catalyst for selective hydrogenation; the polymer solution after polymerization was transferred to a hydrogenated vessel at 70° C., and the prepared catalyst was added therein, wherein the pressure was boosted to 1.8 MPa with hydrogen while fully stirring, and continuously maintained with hydrogen until hydrogenation is completed.

(3) Hydrogenated polymer washing process: same as the washing process in Example 1.

(4) Thermal cross-linking reaction: the elastic material was molded at 240° C. for 30 minutes, and the obtained material was no longer dissolved in toluene (only swelling occurred), indicating that a cross-linking reaction had occurred.

The ¹H-NMR spectra of the elastic material before and after hydrogenation show that the residual double bonds of the butadiene monomer unit after selective catalytic hydrogenation have been completely saturated, and the degree of hydrogenation is 100%.

### Example 7 (Sample Code: RILX-005)

A thermally crosslinkable triblock polymer elastic material: a product of (styrene-co-4VBCB)-poly(isoprene-co-styrene-co-4VBCB)-poly(styrene-co-4VBCB) after selective hydrogenation has the following molecular structure:

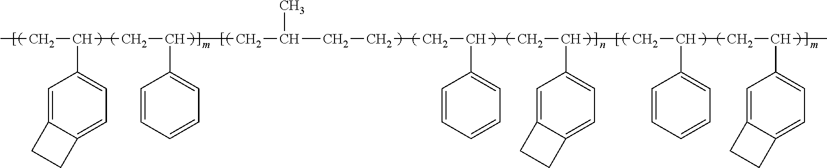 [SMILES: CCC(CC(CC(C)CCCC(CC(CC(CC(C)c1ccc2c(c1)CC2)c1ccccc1)c1ccc2c(c1)CC2)c1ccccc1)c1ccccc1)c1ccc2c(c1)CC2]

Its preparation method is as follows:

(1) Polymerization process: a styrene/4VBCB mixture (the weight of 4VBCB is 2% of the weight of the mixture) was prepared in advance; 450 mL solvent of cyclohexane (water content is 10 ppm) was added into the polymerization vessel, and heated up to 70° C.; styrene/4VBCB mixture (5.5 mL) and 0.10 mL n-butyllithium (1.6 M n-hexane solution) were added in sequence and reacted for 15 minutes; 4.1 mL isoprene and 1.2 mL styrene/4VBCB mixture were added in sequence, and then 12 mL styrene/4VBCB mixture and 7 mL isoprene were added in sequence; then 30 mL isoprene was added therein at a constant speed in 18 minutes for reaction for 30 minutes; 5.5 mL styrene/4VBCB mixture was added therein for reaction for 20 minutes, and then isopropanol was added therein to terminate the polymerization reaction.

(2) Selective catalytic hydrogenation process: 1.0 g of nickel 2-ethylhexanoate was added into a single-necked flask, and dissolved with 57 mL of cyclohexane, then 8.4 mL of triisobutylaluminum (1.1 M toluene solution) was slowly dropped therein and mixed well, which mixture is the catalyst for selective hydrogenation; the polymer solution after polymerization was transferred to a hydrogenated vessel at 70° C., and the prepared catalyst was added therein, wherein the pressure was boosted to 1.8 MPa with hydrogen while fully stirring, and continuously maintained with hydrogen until hydrogenation is completed.

(3) Hydrogenated polymer washing process: same as the washing process in Example 1.

(4) Thermal cross-linking reaction: the elastic material was molded at 240° C. for 20 minutes, and the obtained material was no longer dissolved in toluene (only swelling occurred), indicating that a cross-linking reaction had occurred.

The ¹H-NMR spectra of the elastic material before and after hydrogenation show that the residual double bonds of the isoprene monomer unit after selective catalytic hydrogenation have been completely saturated, and the degree of hydrogenation is 100%.

### Example 8

A thermally crosslinkable triblock elastic material: (styrene-co-4VBCB)-poly(isoprene)-poly(styrene-co-4VBCB) after selective hydrogenation has the following molecular structure:

(A-B)₃—Si—CH₃

Wherein, A represents the copolymer block of styrene and thermally crosslinking monomer, B represents the polyisoprene block after hydrogenation, A-B represents the single-arm diblock copolymer, (A-B)₃ represents that there are three such single-arm diblock copolymers attached to the silicon atom (the A block is at the outer end and the B block is attached to the silicon atom). The molecular structure of the A-B single-arm diblock copolymer is as follows:

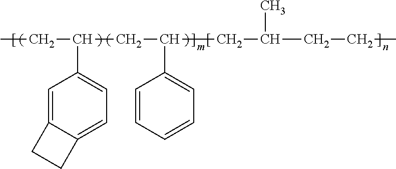

Its preparation method is as follows:

(1) Polymerization process: a styrene/4VBCB mixture (the weight of 4VBCB is 2% of the weight of the mixture) was prepared in advance; 1000 mL solvent of cyclohexane (water content is 10 ppm) was added into the polymerization vessel, and heated up to 70° C.; styrene/4VBCB mixture (33 mL) and 0.75 mL n-butyllithium (1.6 M n-hexane solution) were added in sequence and reacted for 15 minutes; 104 mL isoprene was added therein for reaction for 30 minutes; 0.054 g methyltrimethoxysilane was added therein for reaction for 60 minutes, and then isopropanol was added therein to terminate the polymerization reaction.

(2) Selective catalytic hydrogenation process: 1.25 g of nickel 2-ethylhexanoate was added into a single-necked flask, and dissolved with 57 mL of cyclohexane, then 9.3 mL of triisobutylaluminum (1.1 M toluene solution) was slowly dropped therein and mixed well, which mixture is the catalyst for selective hydrogenation; the polymer solution after polymerization was transferred to a hydrogenated vessel at 70° C., and the prepared catalyst was added therein, wherein the pressure was boosted to 1.8 MPa with hydrogen while fully stirring, and continuously maintained with hydrogen until hydrogenation is completed.

(3) Hydrogenated polymer washing process: same as the washing process in Example 1.

(4) Thermal cross-linking reaction: the elastic material was molded at 240° C. for 20 minutes, and the obtained material was no longer dissolved in toluene (only swelling occurred), indicating that a cross-linking reaction had occurred.

### Example 9 (Sample Code: T210109)

A thermally crosslinkable triblock polymer elastic material: a product of polystyrene-polybutadiene-polystyrene before selective hydrogenation has the following molecular structure:

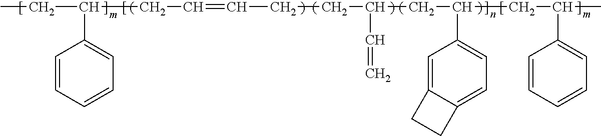

Its preparation method is as follows:

The reaction temperature was 60° C. 200 mL cyclohexane was added to the reaction vessel, and then 0.1 mL styrene and 0.5 mL THF were added therein with syringe, and thereafter n-butyllithium was added dropwise to remove impurities and show yellow. 3.75 g of styrene and 0.1 mL of n-butyllithium (1.6 M n-hexane solution) were continuously added therein for reaction for 15 minutes. 17.5 g of butadiene solution was added therein for reaction for 30 minutes. 3.75 g styrene was added therein for reaction for 30 minutes. After the reaction was completed, isopropanol was added to terminate the reaction. The added butadiene solution is a mixed solution of butadiene and thermally crosslinking monomer of 4-vinylbenzocyclobutene, and the content of the thermally crosslinking monomer in the butadiene solution is 1.5%.

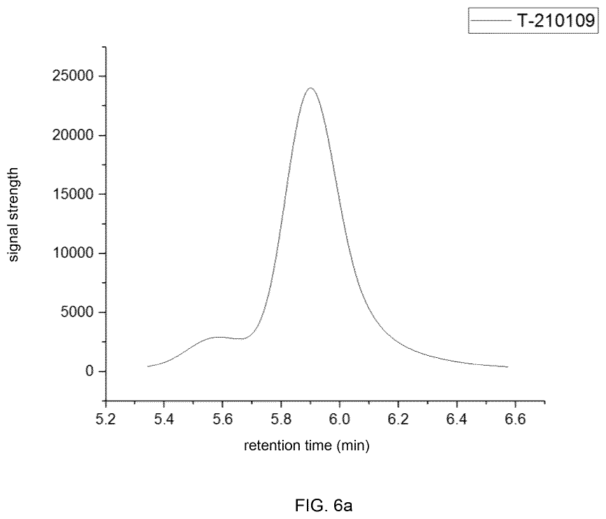

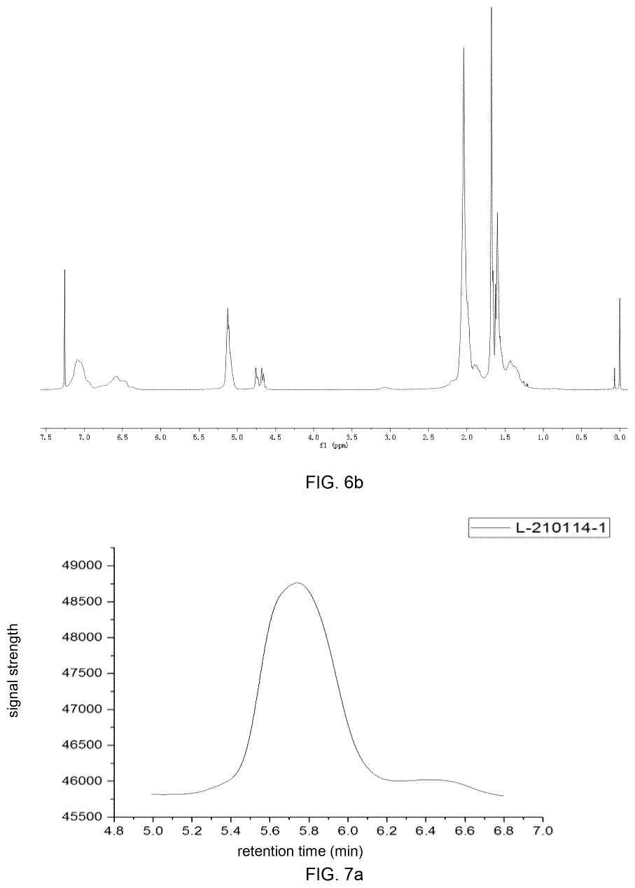

The GPC graph of the reaction product is shown in FIG. 6a, the molecular weight M n of the product is 70000, and the molecular weight distribution is 1.074. The ¹H-NMR spectrum of the reaction product is shown in FIG. 6b.

The reaction product is subjected to catalytic hydrogenation, hydrogenated polymer washing and thermal crosslinking to obtain the elastic material.

### Example 10 (Sample Code: L-210114-1)

A thermally crosslinkable random triblock copolymer, the styrene in the middle and two ends thereof all contains crosslinking agent, and the molecular structure thereof before selective hydrogenation is as follows:

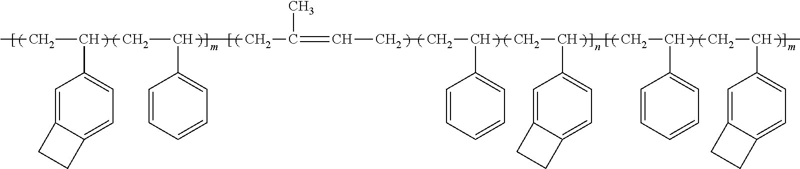

Its preparation method is as follows:

The reaction temperature was 60° C. 200 mL cyclohexane was added to the reaction vessel, and then 0.2 mL styrene solution and 0.5 mL THF were added therein with syringe, and thereafter n-butyllithium was added dropwise to remove impurities and show yellow. 3.75 g of styrene solution and 0.2 mL of n-butyllithium (0.32 M n-hexane solution) were continuously added therein for reaction for 15 minutes. 0.525 g of styrene solution and 3.675 g isoprene were added therein for reaction for 15 minutes. Then 4.725 g styrene solution and 11 g isoprene were added therein for reaction for 20 minutes. 3.75 g styrene solution was added therein for reaction for 15 minutes. After the reaction was completed, isopropanol was added to terminate the reaction. The added styrene solution is a mixed solution of styrene and thermally crosslinking monomer of 4-vinylbenzocyclobutene, and the content of the thermally crosslinking monomer in the styrene solution is 3%.

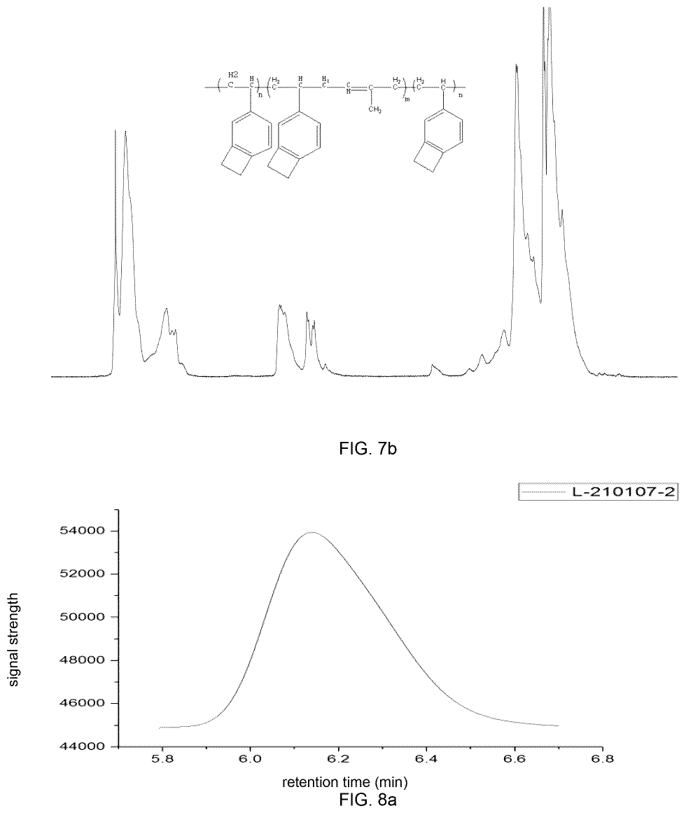

The GPC graph of the reaction product is shown in FIG. 7a, the molecular weight M n of the product is 96000, and the molecular weight distribution is 1.06. The ¹H-NMR spectrum of the reaction product is shown in FIG. 7b.

The reaction product is subjected to catalytic hydrogenation, hydrogenated polymer washing and thermal crosslinking to obtain the elastic material.

### Example 11 (Sample Code: L-210107-2)

A thermally crosslinkable random triblock copolymer, the styrene in the middle and two ends thereof all contains crosslinking agent, and the molecular structure thereof before selective hydrogenation is as follows:

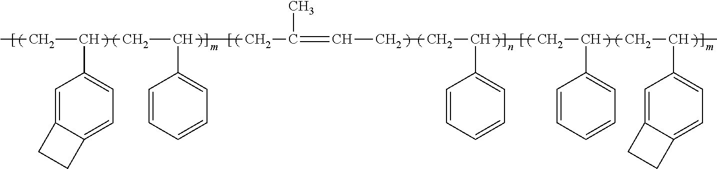

Its preparation method is as follows:

The reaction temperature was 60° C. 200 mL cyclohexane was added to the reaction vessel, and then 0.2 mL styrene solution and 0.5 mL THF were added therein with syringe, and thereafter n-butyllithium was added dropwise to remove impurities and show yellow. 3.75 g of styrene solution and 0.2 mL of n-butyllithium (0.32 M n-hexane solution) were continuously added therein for reaction for 15 minutes. 0.525 g of styrene and 3.675 g isoprene were added therein for reaction for 15 minutes. Then 4.725 g styrene and 11 g isoprene were added therein for reaction for 20 minutes. 3.75 g styrene solution was added therein for reaction for 15 minutes. After the reaction was completed, isopropanol was added to terminate the reaction. The added styrene solution is a mixed solution of styrene and thermally crosslinking monomer of 4-vinylbenzocyclobutene, and the content of the thermally crosslinking monomer in the styrene solution is 3%.

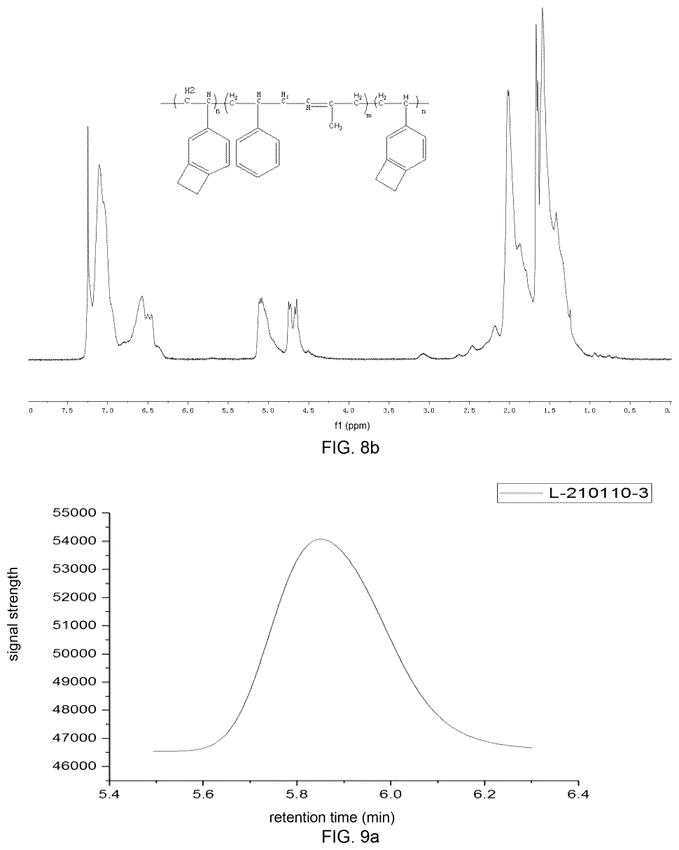

The GPC graph of the reaction product is shown in FIG. 8a, the molecular weight M n of the product is 42000, and the molecular weight distribution is 1.079. The ¹H-NMR spectrum of the reaction product is shown in FIG. 8b.

The reaction product is subjected to catalytic hydrogenation, hydrogenated polymer washing and thermal crosslinking to obtain the elastic material.

### Example 12 (Sample Code: L-210110-3)

A thermally crosslinkable SIS polymer, both styrene and isoprene thereof contain crosslinking agent, and the molecular structure thereof before selective hydrogenation is as follows:

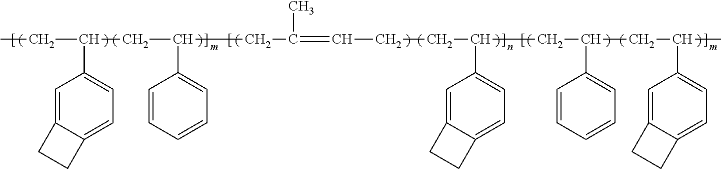

Its preparation method is as follows:

The reaction temperature was 60° C. 200 mL cyclohexane was added to the reaction vessel, and then 0.2 mL styrene solution and 0.5 mL THF were added therein with syringe, and thereafter n-butyllithium was added dropwise to remove impurities and show yellow. 3.75 g of styrene solution and 0.4 mL of n-butyllithium (0.32 M n-hexane solution) were continuously added therein for reaction for 15 minutes. 17.5 g of isoprene solution was added therein for reaction for 40 minutes. 3.75 g styrene solution was added therein for reaction for 15 minutes. After the reaction was completed, isopropanol was added to terminate the reaction. The isoprene solution is a mixed solution of isoprene and thermally crosslinking monomer of 4-vinylbenzocyclobutene, and the content of the thermally crosslinking monomer in the isoprene solution is 1.5%. The styrene solution is a mixed solution of styrene and thermally crosslinking monomer of 4-vinylbenzocyclobutene, and the content of the thermally crosslinking monomer in the styrene solution is 3%.

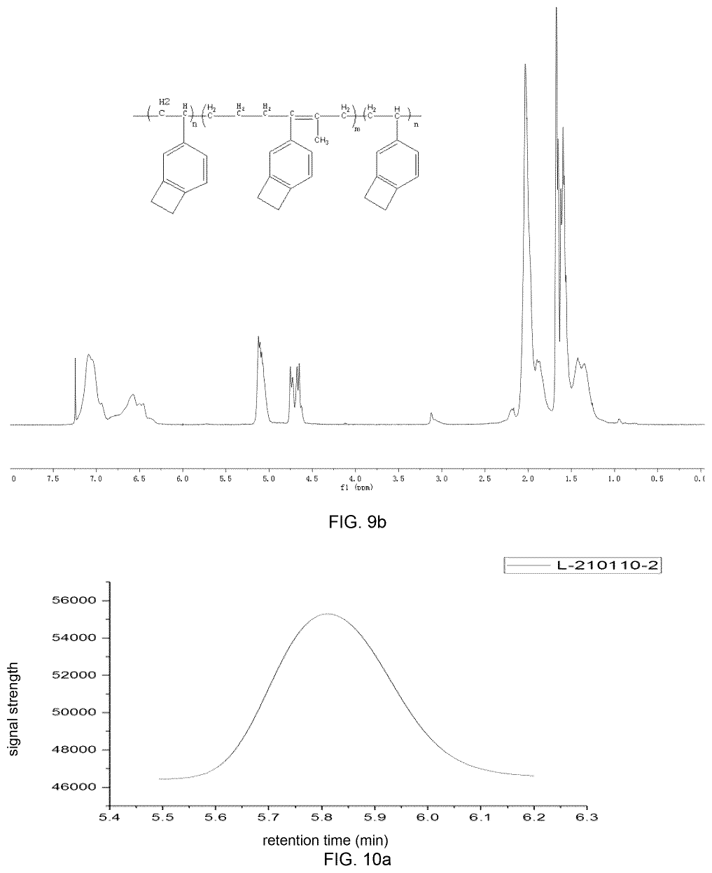

The GPC graph of the reaction product is shown in FIG. 9a, the molecular weight M n of the product is 75000, and the molecular weight distribution is 1.061. The ¹H-NMR spectrum of the reaction product is shown in FIG. 9b.

The reaction product is subjected to catalytic hydrogenation, hydrogenated polymer washing and thermal crosslinking to obtain the elastic material.

### Example 13 (Sample Code: L-210110-2)

A thermally crosslinkable SIS polymer, styrene thereof contains no crosslinking agent, all isoprene thereof contains crosslinking agent, and the molecular structure thereof before selective hydrogenation is as follows:

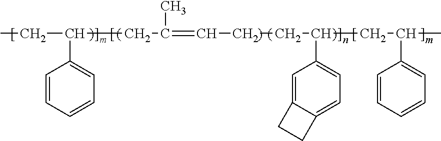

Its preparation method is as follows:

The reaction temperature was 60° C. 200 mL cyclohexane was added to the reaction vessel, and then 0.2 mL styrene and 0.5 mL THF were added therein with syringe, and thereafter n-butyllithium was added dropwise to remove impurities and show yellow. 3.75 g of styrene and 0.4 mL of n-butyllithium (0.32 M n-hexane solution) were continuously added therein for reaction for 15 minutes. 17.5 g of isoprene solution was added therein for reaction for 40 minutes. 3.75 g styrene was added therein for reaction for 15 minutes. After the reaction was completed, isopropanol was added to terminate the reaction. The isoprene solution is a mixed solution of isoprene and thermally crosslinking monomer of 4-vinylbenzocyclobutene, and the content of the thermally crosslinking monomer in the isoprene solution is 1.5%.

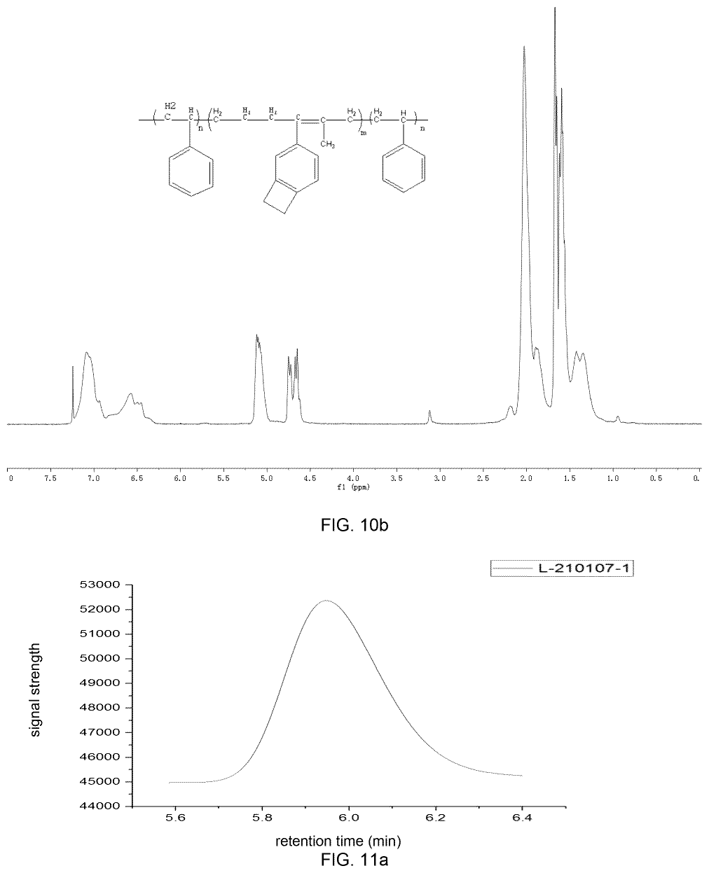

The GPC graph of the reaction product is shown in FIG. 10a, the molecular weight M n of the product is 85000, and the molecular weight distribution is 1.043. The ¹H-NMR spectrum of the reaction product is shown in FIG. 10b.

The reaction product is subjected to catalytic hydrogenation, hydrogenated polymer washing and thermal crosslinking to obtain the elastic material.

### Example 14 (Sample Code: L-210107-1)

A heat-crosslinkable random triblock copolymer, the styrene at two ends thereof contains no crosslinking agent, the styrene in the middle segment thereof contains crosslinking agent, and the molecular structure thereof before selective hydrogenation is as follows:

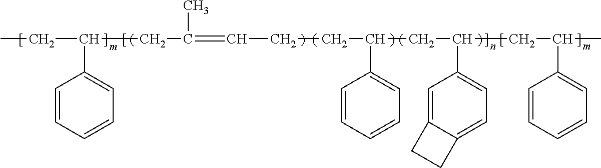

Its preparation method is as follows:

The reaction temperature was 60° C. 200 mL cyclohexane was added to the reaction vessel, and then 0.2 mL styrene and 0.5 mL THF were added therein with syringe, and thereafter n-butyllithium was added dropwise to remove impurities and show yellow. 3.75 g of styrene solution and 0.5 mL of n-butyllithium (0.32 M n-hexane solution) were continuously added therein for reaction for 15 minutes. 0.525 g of styrene solution and 3.675 g isoprene were added therein for reaction for 10 minutes. Then 4.725 g styrene solution and 11 g isoprene were added therein for reaction for 20 minutes. 3.75 g styrene was added therein for reaction for 15 minutes. After the reaction was completed, isopropanol was added to terminate the reaction. The added styrene solution for the middle segment is a mixed solution of styrene and thermally crosslinking monomer of 4-vinylbenzocyclobutene, and the content of the thermally crosslinking monomer in the styrene solution is 3%.

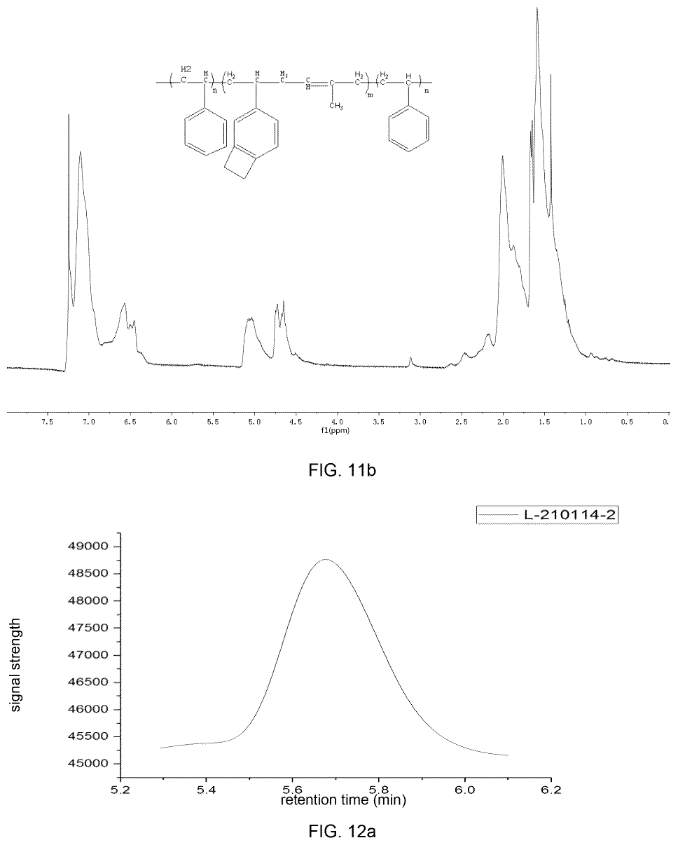

The GPC graph of the reaction product is shown in FIG. 11a, the molecular weight M n of the product is 60000, and the molecular weight distribution is 1.09. The ¹H-NMR spectrum of the reaction product is shown in FIG. 11b.

The reaction product is subjected to catalytic hydrogenation, hydrogenated polymer washing and thermal crosslinking to obtain the elastic material.

### Example 15 (Sample Code: L-210114-2)

A thermally crosslinkable SIS polymer containing 10% isoprene at two ends, both styrene and middle isoprene thereof contain crosslinking agent, and the molecular structure thereof before selective hydrogenation is as follows:

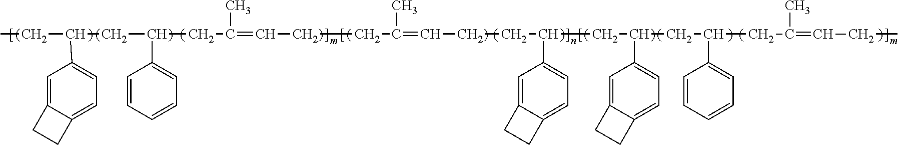

Its preparation method is as follows:

The reaction temperature was 60° C. 200 mL cyclohexane was added to the reaction vessel, and then 0.2 mL styrene solution and 0.5 mL THF were added therein with syringe, and thereafter n-butyllithium was added dropwise to remove impurities and show yellow. 3.375 g styrene solution and 0.375 g isoprene solution were continuously added therein at the same time, and 0.2 mL (0.32 M n-hexane solution) n-butyllithium was added therein for reaction for 15 minutes. 17.5 g of isoprene solution was added therein for reaction for 40 minutes. 3.375 g styrene solution and 0.375 g isoprene solution were added therein at the same time for reaction for 15 minutes. After the reaction was completed, isopropanol was added to terminate the reaction. The styrene solution for the two ends is a mixed solution of styrene and thermally crosslinking monomer of 4-vinylbenzocyclobutene, and the content of the thermally crosslinking monomer in the styrene solution is 3%. The isoprene solution for the middle segment is a mixed solution of isoprene and thermally crosslinking monomer of 4-vinylbenzocyclobutene, and the content of the thermally crosslinking monomer in the isoprene solution is 1.5%.

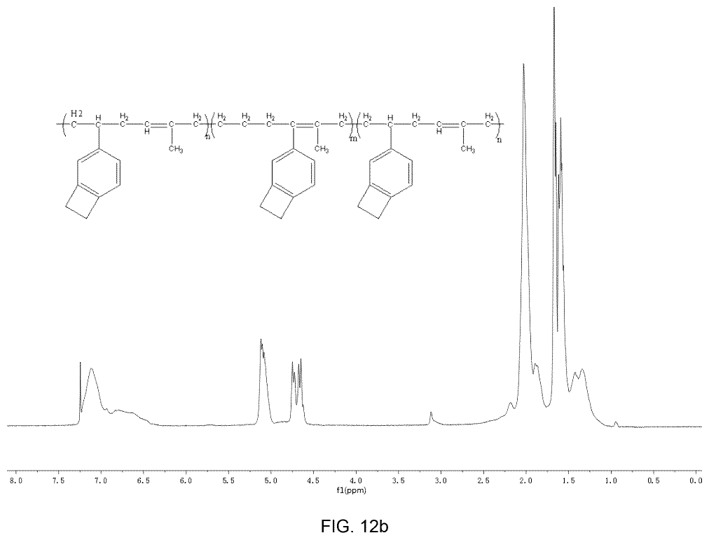

The GPC graph of the reaction product is shown in FIG. 12a, the molecular weight M n of the product is 107000, and the molecular weight distribution is 1.046. The ¹H-NMR spectrum of the reaction product is shown in FIG. 12b.

The reaction product is subjected to catalytic hydrogenation, hydrogenated polymer washing and thermal crosslinking to obtain the elastic material.

### Example 16

The thermally crosslinked products prepared in Examples 1 to 7 of the present invention were tested regarding the basic structure and tensile property, and the results are shown in Table 1. (Note: the molecular weight and molecular weight distribution are the data of the samples before hydrogenation, and the molecular weight increases slightly after hydrogenation).

TABLE 1
Molecular structure parameters and tensile properties of the samples

| | | | | | Content of | Number - | | | | |
|---|---|---|---|---|---|---|---|---|---|---|
| | | | | Content of | polystyrene | average | Molecular | degree of | | |
| | | Content of | Content of | polystyrene | in rubber | Molecular | weight | hydroge- | Tensile | breaking |
| Example | Sample | styrene | 4VBCB | at two ends | phase | Weight | distri- | nation | strength | elongation |
| # | code | (%) | (%) | (%) | (%) | (Dalton) | bution | (%) | (MPa) | (%) |
| 1 | RBHX-001 | 60 | 1.2 | 0 | 60 | 94800 | 1.09 | 100 | 28.2 | 280 |
| 2 | RIHX-003 | 60 | 1.2 | 0 | 60 | 57700 | 1.08 | 95.8 | 26.4 | 300 |
| 3 | RBLX-002 | 47.5 | 0.95 | 25 | 30 | 10400 | 1.07 | 100 | 21.7 | 640 |
| 4 | ILX-001 | 30 | 0.6 | 30 | 0 | 15300 | 1.07 | 95.2 | 21.8 | 600 |
| 5 | RILX-004 | 58 | 1.16 | 30 | 40 | 13400 | 1.07 | ~100 | 14.5 | 560 |
| 6 | BLX-001 | 25 | 0.5 | 25 | 0 | 13020 | 1.06 | 100 | 12.3 | 1120 |
| 7 | RILX-005 | 44 | 0.88 | 20 | 30 | 14780 | 1.07 | 100 | 10.6 | 640 |

### Example 17 In Vitro Accelerated Test of Biological Stability

An in vitro accelerated test of biological stability is to place the sample in boiling concentrated nitric acid (65%), as nitric acid is not only a strong acid but also a strong oxidant [U.S. Pat. No. 6,102,939]. For the sake of experimental safety, the test temperature in this example is room temperature, and the sample and concentrated nitric acid are mixed for 6 hours by a Teflon-coated rotor and a magnetic stirrer (unless otherwise specified).

The elastic materials prepared in Examples 1-3 of the present invention and other elastic materials such as SIBS, SEBS, SEPS, polyolefin elastomer, polyolefin block polymer, polyurethane (including polycarbonate polyurethane PCU and polyether polyurethane PEU) and biological valve material all used the above-mentioned method to carry out the in vitro accelerated test of biological stability, and the test results are shown in Table 2.

TABLE 2
In vitro accelerated test results of biological stability

| | | | | Change in |
|---|---|---|---|---|
| Sample | | | | tensile |
| name | Sample description | Dissolution time | Phenomenon observed | strength |
| SIBS | Polystyrene- | insoluble | no change | basically no |
| | polyisobutylene- | | | change |
| | polystyrene block polymer | | | |
| polyolefin | Copolymer of ethylene | insoluble | no change | decreased |
| elastomer | and 1-octene | | | about 10% |
| polyolefin | The hard segment is | insoluble | no change | basically no |
| block | crystallized polyethylene, | | | change |
| polymer | and the soft segment is a | | | |
| | random copolymer of | | | |
| | ethylene and octene | | | |
| SEBS | Polystyrene- | insoluble | no change | basically no |
| | poly(ethylene-co- | | | change |
| | butylene)-polystyrene | | | |
| | block polymer | | | |
| SEPS | Polystyrene- | insoluble | The colour changed into | decreased |
| | poly(ethylene-co- | | yellow, but the shape did | about 20% |
| | propylene)-polystyrene | | not change | |
| | block copolymer | | | |
| HW010 | Hydrogenated styrene- | insoluble | no change | basically no |
| | based thermoplastic | | | change |
| | elastomer (Example 2) | | | |
| RBLX-002 | Cross-linked polymer of | insoluble | no change | no change |
| | polymer capable of | | | |
| | forming elastic material by | | | |
| | thermal crosslinking | | | |
| | (Example 3) | | | |
| ILX-001 | Elastic material | insoluble | The colour changed into | decreased |
| | (Example 4) | | yellow, but the shape did | about 20% |
| | | | not change | |
| BLX-001 | Elastic material | insoluble | no change | decreased |
| | (Example 6) | | | about 10% |
| RILX-005 | Elastic material | insoluble | no change | decreased |
| | (Example 7) | | | about 10% |
| PCU | Polycarbonate | After 1 hour, | After 1 hour, the sample | completely |
| | polyurethane | severe | deteriorated severely | losing elasticity |
| | | embrittlement | (the sample completely | |
| | | occurred, so the | lost its elasticity and | |
| | | experiment | became brittle) | |
| | | terminated. | | |
| PEU | Polyether polyurethane | Dissolved | After 30 seconds, the | The sample |
| | | completely after | sample turned pink, and | was dissolved |
| | | about 35 minutes | after 20 minutes, the | |
| | | | sample started to | |
| | | | dissolve | |
| biological | porcine pericardium | The surface was | crimping, darkening, | Greatly |
| valve | | depressed | depressed surface | reduced |
| material | | | | (cannot be |
| | | | | measured due |
| | | | | to product |
| | | | | deformation) |

It can be seen from the test results in Table 2 that polyether polyurethane was completely eroded by concentrated nitric acid in about 35 minutes; polycarbonate polyurethane completely lost its elasticity although it has not been eroded, indicating that the molecular structure, especially the soft segment, has undergone serious structural changes; other elastomers (including SIBS, polyolefin elastomer, polyolefin block polymer, styrene-based thermoplastic elastomers SEBS and SEPS, Example 6 and Example 7 of the present invention) were obviously much more stable, although SEPS turned yellow, all samples had no change in shape and basically kept the rubber elasticity (except for the SEPS sample, the rubber elasticity of which was basically unchanged or decreased by only 10%), and the biological valve material (porcine pericardium) was crimpled and discolored, and had many small depressions on the surface, while the strength was greatly reduced.

Based on the test biological stability, although polycarbonate polyurethane is significantly better than polyether polyurethane, these two polyurethane samples are far inferior to other hydrocarbon polymer-based elastic materials (including SEBS, SEPS, polyethylene-based copolymer elastomer, polyethylene-based polyolefin block elastomer, polyisobutylene-based SIBS, and the thermally crosslinked elastic material of the present invention). This indicates that the elastic material prepared according to the present invention has better biological stability than the biological valve material and polyurethane materials.

### Example 18

A hydrogenated styrene-based elastomer (sample code HW009), with a styrene content of 42%, has the following molecular structure:

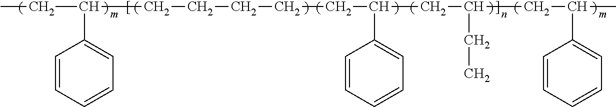

Then it was placed in a mold with a thickness of 0.1 mm, and molded at 240° C. for 30 minutes to prepare the polymer valve.

### Example 19

A hydrogenated styrene-based elastomer (sample code HW010), with a styrene content of 58%, has the following molecular structure:

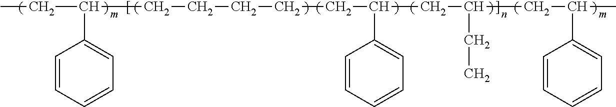

Then it was placed in a mold with a thickness of 0.1 mm, and molded at 240° C. for 30 minutes to prepare the polymer valve.

### Example 20 Blood Compatibility Detection

HW010:

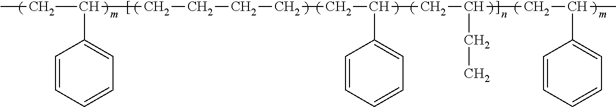

HW014: Commercial Product Dow Polyolefin Elastomer Engage 8137 (Copolymer of Ethylene and 1-Octene)

HZ009:

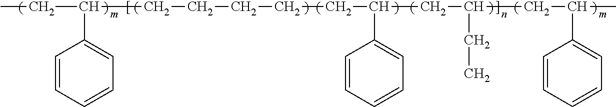

Taking the biological valve material as a control, the blood compatibility tests were carried out on the three polymer materials (HW010, HW014 and HZ009). HW010 is a hydrogenated styrene-based thermoplastic elastomer HSBC; HW014 is a polyolefin elastomer; HZ009 is a cross-linkable SIBS (XSIBS) material. Examples 1-8 all use cross-linkable HSBC (XHSBC) materials, and the chemical compositions thereof are similar to HW010, so the test results of HW010 can be analogized to the polymer materials in Examples 1-8.

As the blood compatibility test results depend on the properties of the surface materials, the test results of the polymers can be analogized to the test results of the composite materials according to the examples of the present invention.

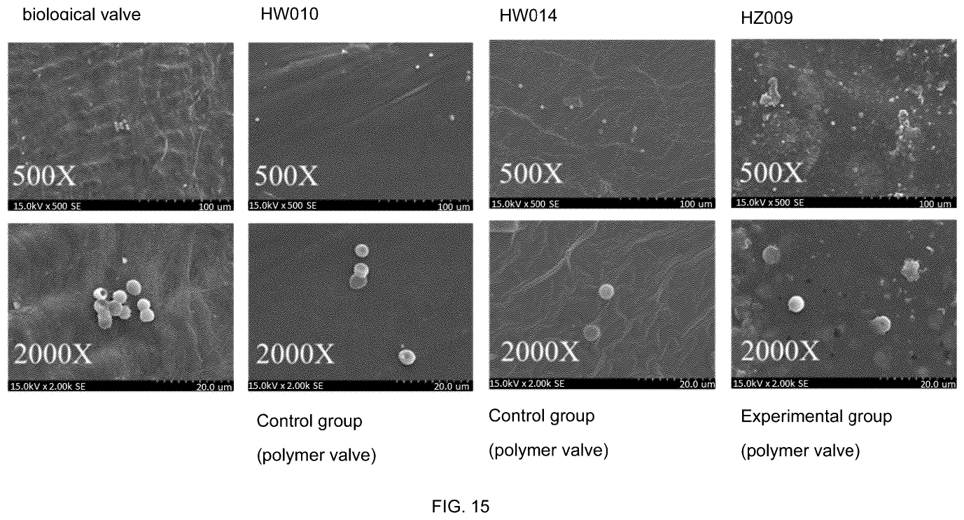

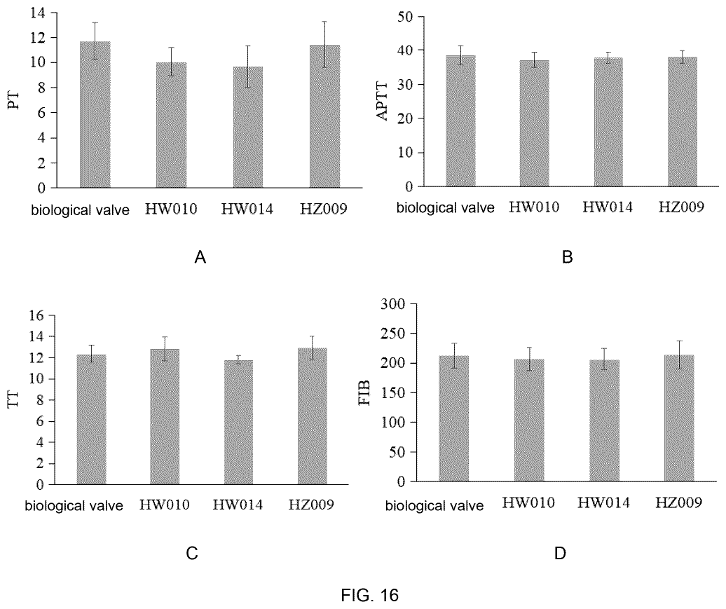

FIG. 14 shows the results of the platelet adhesiveness test, FIG. 15 shows the results of the blood adhesiveness test, and FIG. 16 shows the results of blood coagulation four items. These test results show that there is no significant difference in blood compatibility between these polymer materials and biological valve material. It can be concluded that the thermally crosslinkable polymer elastic materials for making the heart valve prostheses according to the examples of the present invention have no significantly difference in the blood compatibility from the biological valve, and they will not cause blood coagulation problems when used as heart valves like the biological valve.

### Example 21 Anti-Calcification Performance Detection

Taking the biological valve material as a control, the three polymer materials (HW010, HW014 and HZ009) were implanted into rats for 90 days to conduct calcification experiments. HW010 is a HSBC; HW014 is a polyolefin elastomer; HZ009 is a cross-linkable SIBS (XSIBS) material. Examples 1-8 all use cross-linkable HSBC (XHSBC) materials, and the chemical compositions thereof are similar to HW010, so the test results of HW010 can be analogized to the polymer materials in Examples 1-8.

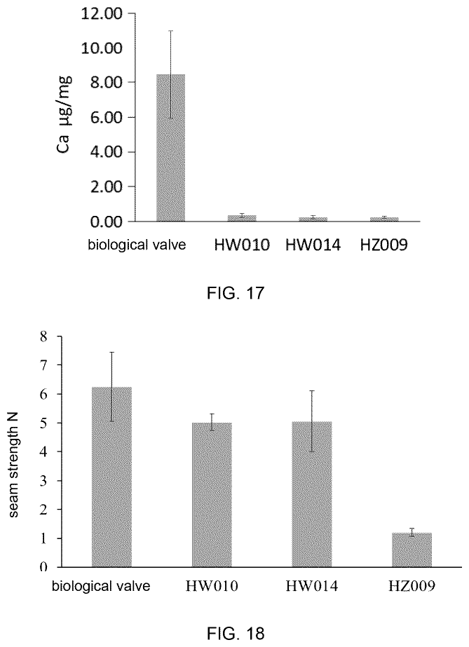

FIG. 17 shows the calcification results, which shows that the calcification degrees of these polymer materials (HW010, HW014, HZ009) are significantly lower than that of the biological valve material, so it can be concluded that the thermally crosslinkable polymer elastic materials for making the heart valve prostheses according to the examples of the present invention have better anti-calcification performance in the organism than the biological valve, and thus the heart valve prostheses made of these materials can overcome the problem that the biological valve is prone to calcification.

In summary, according to the test results of the polymers capable of forming elastic materials by thermal crosslinking disclosed herein, it can be concluded that the materials can be used for making heart valve prostheses for implantation into the human body through thoracotomy or minimally invasive replacement surgery through small incisions.

As the calcification results depend on the properties of the surface materials, the test results of the polymers can be analogized to the test results of the composite materials in the examples of the present invention.

### Example 22 Seam Strength Test

Taking the biological valve material as a control, three polymer materials (HW010, HW014 and HZ009) were prepared into thin films with a thickness of about 0.15 mm by hot pressing, and then the seam strength was tested by simulation experiments. HW010 is a HSBC; HW014 is a polyolefin elastomer; HZ009 is a cross-linkable SIBS (XSIBS) material. FIG. 18 shows the results of seam strength, which shows that the seam strength of polymer materials HW010 and HW014 is close to that of the biological valve material, while the seam strength of HZ009 is obviously lower. This indicates that the polymer materials of the present invention have the necessary seam strength, and can be sewn into qualified heart valve prostheses product like the biological valve material.

The technical features of the above-mentioned examples can be combined arbitrarily. To make the description concise, all possible combinations of the technical features in the above-mentioned examples are not described. However, as long as there is no contradiction in the combinations of these technical features, such combinations should be considered as falling within the scope of the specification.

The above-mentioned examples only represent several embodiments of the present invention, and the description thereof is relatively specific and detailed, but it should not be construed as limiting the protection scope of the invention. It should be noted that the skilled person in the art can make several modifications and developments without departing from the inventive concept of the present invention, which all fall into the protection scope of the present invention. Therefore, the scope of protection of the present invention should be based on the appended claims.

1. A polymer capable of forming elastic material by thermal crosslinking and synthesized by anionic polymerization, the elastic material is a saturated block copolymer, comprising polymer A as hard segment and polymer B as soft segment, with a chemical formula of:
(Aₘ)ᵢ(Bₙ)ⱼ(A_{f})ₖ or (Aₘ−Bₙ)ₚX(Bₙ−A_{f})_{q};
wherein compositions of polymers A at two ends of the polymer B are independent of each other;
the polymer A is a polymer formed by polymerization of at least one of vinyl aromatic hydrocarbon and thermally crosslinking monomer, or a polymer formed by copolymerization of at least one of vinyl aromatic hydrocarbon and thermally crosslinking monomer with conjugated diene;
the polymer B is a conjugated diene polymer, or a polymer formed by copolymerization of at least one of vinyl aromatic hydrocarbon and thermally crosslinking monomer with conjugated diene;
at least one of the polymer A and the polymer B contains the thermally crosslinking monomer;
X represents a residual group of a coupling agent after coupling reaction;
subscripts m and f each represents a number of comonomer units of the polymer A, subscript n represents a number of comonomer units of the polymer B, and m, n, f are all integers greater than or equal to 1 and independent of each other;
subscripts i and k each represents a number of blocks of the polymer A, subscript j represents a number of blocks of the polymer B, both i and k are integers greater than or equal to 0, j is an integer greater than or equal to 1, and i, j, k are independent of each other;
subscripts p and q each represents a number of blocks formed by polymerization of the polymer A and the polymer B, and both p and q are integers greater than or equal to 0 and are independent of each other;
the thermally crosslinking monomer has a chemical structural formula of: 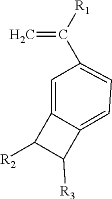
wherein, R₁, R₂ and R₃ are each hydrogen or C₁-C₁₀ alkyl group, and are independent of each other.

2. The polymer capable of forming elastic material by thermal crosslinking and synthesized by anionic polymerization according to claim 1, wherein at least one of the polymer A and the polymer B contains the vinyl aromatic hydrocarbon.

3. The polymer capable of forming elastic material by thermal crosslinking and synthesized by anionic polymerization according to claim 1, wherein the polymer A is the polymer formed by polymerization of at least one of vinyl aromatic hydrocarbon and thermally crosslinking monomer, or the polymer formed by copolymerization of at least one of vinyl aromatic hydrocarbon and thermally crosslinking monomer with conjugated diene;
the polymer B is the conjugated diene polymer, or the polymer formed by copolymerization of at least one of vinyl aromatic hydrocarbon and thermally crosslinking monomer with conjugated diene; and
when the polymer A is a polymer formed by polymerization of vinyl aromatic hydrocarbon and thermally crosslinking monomer, the polymer B is the polymer formed by copolymerization of at least one of vinyl aromatic hydrocarbon and thermally crosslinking monomer with conjugated diene.

4. The polymer capable of forming elastic material by thermal crosslinking and synthesized by anionic polymerization according to claim 1, wherein the polymer A is the polymer formed by polymerization of at least one of vinyl aromatic hydrocarbon and thermally crosslinking monomer, and at least one segment of the polymer A of the elastic material contains the thermally crosslinking monomer.

5. The polymer capable of forming elastic material by thermal crosslinking and synthesized by anionic polymerization according to claim 1, wherein the polymer A is the polymer formed by polymerization of at least one of vinyl aromatic hydrocarbon and thermally crosslinking monomer, or the polymer formed by copolymerization of at least one of vinyl aromatic hydrocarbon and thermally crosslinking monomer with conjugated diene; and
the polymer B is the polymer formed by copolymerization of at least one of vinyl aromatic hydrocarbon and thermally crosslinking monomer with conjugated diene.

6. The polymer capable of forming elastic material by thermal crosslinking and synthesized by anionic polymerization according to claim 1, wherein the polymer A is the polymer formed by polymerization of at least one of vinyl aromatic hydrocarbon and thermally crosslinking monomer, and at least one segment of the polymer A of the elastic material contains the thermally crosslinking monomer; and
the polymer B is the polymer formed by copolymerization of at least one of vinyl aromatic hydrocarbon and thermally crosslinking monomer with conjugated diene.

7. The polymer capable of forming elastic material by thermal crosslinking and synthesized by anionic polymerization according to claim 1, wherein the polymer A is a polymer of the vinyl aromatic hydrocarbon, or a copolymer of the vinyl aromatic hydrocarbon and the thermally crosslinking monomer, and at least one segment of the polymer A of the elastic material contains the thermally crosslinking monomer; and
the polymer B is the polymer formed by copolymerization of at least one of vinyl aromatic hydrocarbon and thermally crosslinking monomer with conjugated diene.

8. The polymer capable of forming elastic material by thermal crosslinking and synthesized by anionic polymerization according to claim 1, wherein the polymer A is a copolymer of the vinyl aromatic hydrocarbon and the thermally crosslinking monomer; and
the polymer B is the polymer formed by copolymerization of at least one of vinyl aromatic hydrocarbon and thermally crosslinking monomer with conjugated diene.

9. The polymer capable of forming elastic material by thermal crosslinking and synthesized by anionic polymerization according to claim 1, wherein the vinyl aromatic hydrocarbon is at least one of styrene, α-methylstyrene, 4-methylstyrene, vinylnaphthalene, 1,1-diphenylethene and divinylbenzene; and
wherein the conjugated diene is at least one of isoprene, 1,3-butadiene, 1,3-pentadiene, 4-methylpentadiene, and 2-methylpentadiene.

10. The polymer capable of forming elastic material by thermal crosslinking and synthesized by anionic polymerization according to claim 1, wherein the vinyl aromatic hydrocarbon is styrene, the conjugated diene is at least one of isoprene and 1,3-butadiene, and the thermally crosslinking monomer is 4-vinylbenzocyclobutene.

11. A preparation method for a polymer capable of forming elastic material by thermal crosslinking and synthesized by anionic polymerization, comprising steps of:
(1) performing anionic polymerization of vinyl aromatic hydrocarbon, conjugated diene and thermally crosslinking monomer in solution in presence of anionic polymerization initiator, in an inert gas atmosphere and at a polymerization temperature of −30-150° C.;
wherein amounts of monomer units involved in the polymerization are: a weight percentage of the vinyl aromatic hydrocarbon is 0.01-80%, a weight percentage of the conjugated diene is 20-99.99%, and a weight percentage of the thermally crosslinking monomer is 0.01-30%; and
the thermally crosslinking monomer has a chemical structural formula of: 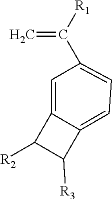
wherein, R₁, R₂ and R₃ are each hydrogen or C₁-C₁₀ alkyl group, and are independent of each other; and
(2) performing catalytic hydrogenation after the polymerization to obtain the polymer capable of forming elastic material by thermal crosslinking.

12. A polymer for heart valve prosthesis that is capable of forming elastic material by thermal crosslinking, the elastic material comprising polymer A as hard segment and polymer B as soft segment which are polymerized to form a copolymer, with a chemical formula of: (Aₘ)ᵢ(Bₙ)ⱼ(A_{f})ₖ; or (Aₘ−Bₙ)ₚX(Bₙ−A_{f})_{q};
wherein compositions of polymers A at two ends of the polymer B are independent of each other;
the polymer A is a polymer formed by polymerization of at least one of vinyl aromatic hydrocarbon and thermally crosslinking monomer, or a polymer formed by copolymerization of at least one of vinyl aromatic hydrocarbon and thermally crosslinking monomer with conjugated diene;
the polymer B is a conjugated diene polymer, or a polymer formed by copolymerization of at least one of vinyl aromatic hydrocarbon and thermally crosslinking monomer with conjugated diene;
at least one of the polymer A and the polymer B contains the thermally crosslinking monomer;
X represents a residual group of a coupling agent after coupling reaction;
subscripts m and f each represents a number of comonomer units of the polymer A, subscript n represents a number of comonomer units of the polymer B, and m, n, f are all integers greater than or equal to 1 and independent of each other;
subscripts i and k each represents a number of blocks of the polymer A, subscript j represents a number of blocks of the polymer B, both i and k are integers greater than or equal to 0, j is an integer greater than or equal to 1, and i, j, k are independent of each other;
subscripts p and q each represents a number of blocks formed by polymerization of the polymer A and the polymer B, and both p and q are integers greater than or equal to 0 and are independent of each other;
the thermally crosslinking monomer has a chemical structural formula of: 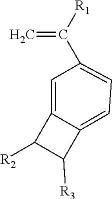
wherein, R₁, R₂ and R₃ are each hydrogen or C₁-C₁₀ alkyl group, and are independent of each other.

13. The polymer for heart valve prosthesis that is capable of forming elastic material by thermal crosslinking according to claim 12, wherein the polymer A is the polymer formed by polymerization of at least one of vinyl aromatic hydrocarbon and thermally crosslinking monomer, or the polymer formed by copolymerization of at least one of vinyl aromatic hydrocarbon and thermally crosslinking monomer with conjugated diene;
the polymer B is the conjugated diene polymer, or the polymer formed by copolymerization of at least one of vinyl aromatic hydrocarbon and thermally crosslinking monomer with conjugated diene; and
when the polymer A is a polymer formed by polymerization of vinyl aromatic hydrocarbon and thermally crosslinking monomer, the polymer B is the polymer formed by copolymerization of at least one of vinyl aromatic hydrocarbon and thermally crosslinking monomer with conjugated diene.

14. The polymer for heart valve prosthesis that is capable of forming elastic material by thermal crosslinking according to claim 12, wherein the polymer A is a copolymer of the vinyl aromatic hydrocarbon and the thermally crosslinking monomer; and
the polymer B is a copolymer of the vinyl aromatic hydrocarbon, the conjugated diene and the thermally crosslinking monomer, or the polymer B is a copolymer of the vinyl aromatic hydrocarbon and the conjugated diene, or B is a copolymer of the conjugated diene and the thermally crosslinking monomer.

15. The polymer for heart valve prosthesis that is capable of forming elastic material by thermal crosslinking according to claim 12, wherein the polymer A is a copolymer of the vinyl aromatic hydrocarbon, the conjugated diene and the thermally crosslinking monomer; and
the polymer B is a copolymer of the vinyl aromatic hydrocarbon, the conjugated diene and the thermally crosslinking monomer, or the polymer B is a copolymer of the vinyl aromatic hydrocarbon and the conjugated diene, or B is a copolymer of the conjugated diene and the thermally crosslinking monomer.

16. The polymer for heart valve prosthesis that is capable of forming elastic material by thermal crosslinking according to claim 12, wherein the polymer A is a polymer of the vinyl aromatic hydrocarbon; and
the polymer B is a copolymer of the vinyl aromatic hydrocarbon, the conjugated diene and the thermally crosslinking monomer or B is a copolymer of the conjugated diene and the thermally crosslinking monomer.

17. The polymer for heart valve prosthesis that is capable of forming elastic material by thermal crosslinking according to claim 12, wherein a molecular weight of the polymer that is capable of forming elastic material by thermal crosslinking is 20000-1000000; and
wherein a tensile strength of the polymer that is capable of forming elastic material by thermal crosslinking is greater than 10 MPa, and a breaking elongation thereof is greater than 300%.

18. A heart valve prosthesis, which is formed by the polymer that is capable of forming elastic material by thermal crosslinking according to claim 12 by thermally crosslinking; and
wherein a thickness of the heart valve prosthesis is 0.02-0.40 mm.

19. An elastic material, which is formed by the polymer according to claim 1 through thermally crosslinking.

20. An interventional device, wherein the elastic material according to claim 19 is applied in the interventional device, and the interventional device is intraocular lens, prosthetic valve, glaucoma drainage tube, lacrimal canalicular plug, medical occluder, artificial disc, artificial joint, artificial ligament, artificial meniscus, vascular graft, cardiac pacemaker, or lead insulator.
